(19) United States
(12) Patent Application Publication
(10) Pub. No.: US 2006/0171947 A1
(43) Pub. Date: Aug. 3, 2006

(54) Serine/threonine hydrolase proteins and screening assays

(75) Inventors: Jeffrey W. Smith, San Diego, CA (US); Steven J. Kridel, San Diego, CA (US); Fumiko T. Axelrod, San Diego, CA (US)

Correspondence Address: Lisa A. Haile, J.D., Ph.D.;DLA PIPER RUDNICK GRAY CARY US LLP, Suite 1100, 4365 Executive Drive, San Diego, CA, 92121-2133, US

(73) Assignee: BURNHAM INSTITUTE

(21) Appl. No.: 11/343,911

(22) Filed: Jan. 30, 2006

Related U.S. Application Data
(63) Continuation of application No. 10/237,271, filed on Sep. 4, 2002.
(60) Provisional application No. 60/317,842, filed on Sep. 6, 2001.

(51) Int. Cl. A61K 39/395; C07H 21/04; C12P 21/06; C12N 9/64

(52) U.S. Cl. 424/145.1; 424/178.1; 435/226; 435/69.1; 435/320.1; 435/325; 530/388.26; 536/23.2

(57) ABSTRACT

Proteins specific for prostate epithelial cells, normal or neoplastic, are identified and used for diagnosis, development of antibodies, and for evaluating drugs that react with the neoplastic specific proteins. Affinity based probes are used that react specifically with the active site to provide a measure of the enzyme activity of the cells. Prostate epithelial neoplastic cells can be used in screening candidate drugs for their effect in changing the proteome profile as to the serine-threonine hydrolase enzymes, using the affinity based probes for determining the profile.

This application claims the benefit of priority under 35 U.S.C. 119(e) of U.S. Ser. No. 60/317,842, filed Sep. 6, 2001, the entire contents of which is incorporated herein by reference.

### BACKGROUND OF THE INVENTION

1. Field of the Invention

The present invention relates generally to serine/threonine hydrolases, and more specifically to compositions and their detection for cellular profiles.

2. Background Information

With the field of genomics in a “mopping up” operation to correct the errors in the genome and to identify differences in sequences in the population, proteomics has newly attracted attention. The advances in combinatorial chemistry allow for the production of large libraries of compounds in amounts that can be tested for biological activity. High throughput screening has galvanized many companies to develop equipment, protocols and reagents to rapidly evaluate large numbers of compounds for biological activity. Such screens can be used to identify affinities for candidate drugs with biological targets such as proteins. To this end, in order to determine whether a target is useful, its function generally must be determined, the pathways in which the target protein acts defined, and the effect of modulating the activity of the target on cellular activity examined.

In many situations, changes in the environment, the state of differentiation of a cell, the nature of the cell, the occurrence of an infection or inflammation, or exposure to or contact with any other agent that can affect the cellular activity is associated with a change in the expression pattern or activity pattern of the proteins in the cell. While a determination of the absolute or relative amount of a particular protein in a cell at a given time can be informative as to the status of the cell, for example, a disease state, a determination of the activity of the protein in the cell at a given time not only can provide diagnostic or prognostic information about the cell, but further can provide a means to manipulate the cell and, therefore, contribute to a therapeutic plan for treating the disease.

Proteins can be in an active or inactive state, and the state of activity (or inactivity) can be a result of modification to the protein such as phosphorylation, dephosphorylation, acetylation, or methylation; formation of a complex with a second protein, which can be the same or different; movement or partitioning to a particular compartment in the cell; and the like. In studying a disease state, information as to the proteome of the cell, i.e., the profile of all of the proteins in the cell, active and inactive proteins can be derived. In particular, the active proteome, which is a profile of all of the proteins in their active form in a cell can be determined.

The identification of proteome profiles allows for a comparison, for example, of proteins in a cell being examined to one or more profiles that are characteristic of normal cells or of one or more cells associated with a diseased state, thus providing a means to diagnose a pathologic or other condition. Furthermore, the proteome profile of a cell be examined, including a cell associated with a disease state in an individual, with the proteome profiles obtained from cells that are known to be susceptible (or refractory) to a particular therapy or combination of therapies, thus providing a means to identify agents that can be useful for rectifying a change associated with the pathology, restoring the cell to its normal phenotype, or killing or otherwise ablating the reproductive capacity of the cell.

Cancer remains a major cause of morbidity and mortality throughout the world, particularly in older individuals. Among men, prostate cancer is particularly prevalent, and the incidence clearly increases with age. Prostate cancer can present as a slowly progressing and relatively mild condition that not require significant treatment, or can present in a very aggressive form that metastasizes to other organs and results in death. While various methods can be used to treat prostate cancer, including surgery, chemotherapy, and radiation therapy, the various treatments that are available can produce significant deleterious side effects, can involve substantial costs, and can vary as to their choice and effectiveness. As such, it would be desirable if markers were available that were predictive as to the manner of treatment, the outcome, and the progress of the disease during treatment. Unfortunately, only a few such markers have been described, and they generally are prognostic of only whether a single type of therapy may be effective. Thus, a need exists to identify markers of diseased cells that can be diagnostic and prognostic, thereby directing the clinician as to which among a variety of potential therapies is most likely to be efficacious. The present invention satisfies this need, and provides additional advantage.

### SUMMARY OF THE INVENTION

Methods and compositions are provided for screening epithelial cells, particularly prostate epithelial cells, for neoplastic activity, for identifying compounds that change the neoplastic activity of the cells or kill the cells, and for staging cancerous cells for their aggressiveness, as well as for suggesting particular modes of treatment. In the event of metastasis, the cancer cells can be identified as derived from prostate cells by the level of target enzyme activity in the cells. Specific proteins also are provided that can be used, for example, in diagnostic assays, for the production of specific antibodies, and for screening compounds for their inhibitory activity. Prostate specific antigen (PSA) in its active state can be assayed for detection of prostate cancer.

The present invention relates to an isolated protein characterized by having an apparent molecular mass of about 70 kDa to 95 kDa; having serine hydrolase activity, which can be inhibited by isoleucine-thiazolidide; being detectable in prostate cancer cells, and reduced or absent in normal prostate cells; and being reactive with a probe, which consists of a fluorophosphonate group linked to a fluorescer or biotin through an alkylene or oxyalkylene group. The isolated protein can be, for example, a dipeptidyl peptidase. The protein can be bound to the probe through an alkylene or oxyalkylene group. The prostate cells can be from any mammal, for example, human prostate cells.

The present invention also relates to an isolated protein characterized by having serine-threonine hydrolase activity; being detectable in prostate cancer cells, and reduced or absent in normal prostate cells; being reactive with a probe consisting of a fluorophosphonate group linked to a fluorescer or biotin through an alkylene or oxyalkylene group; and having an apparent molecular mass of about 48 kDa or about 27 kDa to 28 kDa. For example, the protein can be an acyl Co-A thioesterase having an apparent molecular mass of about 48 kDa, or can be an epoxide hydrolase having an apparent molecular mass of about 27 kDa to 28 kDa. In addition, the present invention further relates to a protein conjugate, which comprises the reaction product of a fatty acid synthase and a probe consisting of a fluorophosphonate group linked to a fluorescer or biotin through an alkylene or oxyalkylene group.

The present invention also relates to method for determining the status of a prostate epithelial cell, wherein the status is indicative of a normal condition, a hyperplastic condition, or a neoplastic condition. Such a method can be performed, for example, by detecting at least three active serine-threonine hydrolases in prostate epithelial cells, wherein the serine-threonine hydrolases are selected from a fatty acid synthase, a dipeptidyl peptidase (DPP) having an apparent molecular mass of about 70 kDa to 95 kDa, a prolyl endopeptidase having an apparent molecular mass of about 71 kDa, a peroxisomal long chain acyl-CoA thioesterase having an apparent molecular mass of about 48 kDa, an epoxide hydrolase having an apparent molecular mass of about 28 kDa, a lysophospholipase-1 having an apparent molecular mass of about 23 kDa, and a protein having an apparent molecular mass of about 60 kDa, wherein the active protein is present in normal neoplastic prostate epithelial cells, and is reduced or absent in neoplastic prostate epithelial cells; wherein the presence of at least three of the serine-threonine hydrolases is indicative of a neoplastic condition. According to such a method, the detecting can be performed, for example, by contacting a lysate of the prostate epithelial cell with a probe consisting of a fluorophosphonate group reactive with an active site of a serine-threonine hydrolase joined to a ligand for binding to a receptor or for fluorescence detection by means of an alkylene or oxyalkylene linker, and detecting specific binding of the probe to a serine-threonine hydrolase. In one embodiment, at least one of the three serine-threonine hydrolases is a DPP other than DPP-IV. In another embodiment, the prostate epithelial cell is a human prostate epithelial cell.

The present invention further relates to a method for identifying a compound effective for treating a prostate epithelial neoplasia. Such a screening assay, can be performed, for example, by determining a level of activity of at least serine-threonine hydrolases in a prostate epithelial cell in the presence and absence of the compound, wherein the serine-threonine hydrolases are selected from a fatty acid synthase, a DPP having an apparent molecular mass of from about 70 kDa to 95 kDa, a prolyl endopeptidase having an apparent molecular mass of about 71 kDa, a peroxisomal long chain acyl-CoA thioesterase having an apparent molecular mass of about 48 kDa, an epoxide hydrolase having an apparent molecular mass of about 28 kDa, and lysophospholipase-1 having an apparent molecular mass of about 23 kDa; and detecting a difference in the level of activity of at least three serine-threonine hydrolases in the presence as compared to the absence of the compound. In one embodiment of the screening assay, at least one of said three serine-threonine hydrolases is a DPP, except that the DPP is not DPP-IV. In another embodiment, the prostate epithelial cell is a human prostate epithelial cell. A screening assay of the invention is particularly amenable to a high throughput format, thereby providing a means to screen, for example, a combinatorial library of small organic molecules, peptides, nucleic acid molecules, and the like.

The present invention also relates to an isolated antibody, which specifically binds a protein selected from a DPP having an apparent molecular mass of about 80 kDa, a DPP having an apparent molecular mass of about 73 kDa; a prolyl endopeptidase having an apparent molecular mass of about 71 kDa, and an epoxide hydrolase having an apparent molecular mass of about 28 kDa, wherein the protein is present in neoplastic prostate epithelial cells, and wherein the protein is not present in normal prostate epithelial cells. In addition, the present invention relates to an isolated antibody that specifically binds a protein conjugate, which comprises a DPP having an apparent molecular mass of about 80 kDa, a DPP having an apparent molecular mass of about 73 kDa; a prolyl endopeptidase having an apparent molecular mass of about 71 kDa, or an epoxide hydrolase having an apparent molecular mass of about 28 kDa, bound to a probe consisting of a fluorophosphonate group linked to a fluorescer or biotin through an alkylene or oxyalkylene group, wherein the antibody specifically binds to the probe component of the protein conjugate, the protein component of the protein conjugate, or an epitope comprising the protein and the probe of the protein conjugate. Accordingly, the present invention further relates to a complex, which includes a protein conjugate, which comprises a protein bound to a probe consisting of a fluorophosphonate group linked to a fluorescer or biotin through an alkylene or oxyalkylene group, wherein the protein is a DPP having an apparent molecular mass of about 80 kDa, a DPP having an apparent molecular mass of about 73 kDa; a prolyl endopeptidase having an apparent molecular mass of about 71 kDa, or an epoxide hydrolase having an apparent molecular mass of about 28 kDa, wherein the protein is present in neoplastic prostate epithelial cells, and is reduced or absent in normal prostate epithelial cells; the complex further comprising an antibody that specifically binds the protein conjugate.

The present invention also provides a complex, which includes a PSA conjugate, which comprises a reaction product of PSA and a probe consisting of a fluorophosphonate group linked to a fluorescer or biotin through an alkylene or oxyalkylene group; and an antibody that specifically binds the PSA conjugate. The antibody can specifically bind the PSA, can specifically bind the fluorescer or biotin, or can specifically bind an epitope comprising PSA and the fluorescer or an epitope comprising PSA and biotin.

The present invention further relates to a method for determining the amount of PSA in an active conformation in a sample. Such a method can be performed, for example, by contacting the sample, a probe consisting of a fluorophosphonate group linked to a fluorescer or biotin through an alkylene or oxyalkylene group, wherein the probe can specifically bind PSA in an active conformation, thereby forming a conjugate comprising PSA in an active conformation, and an antibody, which can specifically bind to the PSA conjugate to form a complex comprising the conjugate and the antibody; and determining the amount of conjugate bound to said antibody, thereby determining the amount in the sample of PSA in an active conformation. The antibody can be specific for PSA, can be specific for a portion of the probe, or can be specific for an epitope formed by the probe and PSA.

The present invention also relates to a method for determining the ratio in a sample of enzymatically active PSA to enzymatically inactive PSA. Such a method can be performed, for example, by contacting the sample with a probe consisting of a fluorophosphonate group linked to a fluorescer or biotin through an alkylene or oxyalkylene group to form a conjugate, wherein the probe can specifically bind enzymatically active PSA; separating the conjugate comprising enzymatically active PSA from the sample using an antibody that specifically binds to the probe, thereby obtaining an immune complex comprising the conjugate and a conjugate-free sample; contacting the conjugate-free sample with an antibody that specifically binds PSA to form an immune complex comprising enzymatically inactive PSA; and determining a ratio of the amount of immune complex comprising the conjugate, which comprises enzymatically active PSA, to the amount of immune complex comprising the PSA, thereby determining a ratio in the sample of enzymatically active PSA to enzymatically inactive PSA.

### BRIEF DESCRIPTION OF THE FIGURES

FIG. 1 shows the effect of DHT on cell proliferation and aggregate serine hydrolase activity. LNCaP cell proliferation was measured in the presence of either 0.1 nM or 100 nM DHT. Cells were plated in 24 well plates in RPMI 1640 with no phenol red and supplemented with charcoal stripped FBS and allowed to adhere overnight. The following day media was replaced, with or without DHT, and cells were grown for six days with media change every second day. Cells were counted using a hemocytometer.

FIG. 2 shows 1) the amino acid and nucleotide sequences for fatty acid synthase; 2) and 3) the amino acid sequences for two dipeptidyl peptidase-like polypeptides; 4) the amino acid and nucleotide sequences for N-acylaminoacyl peptide hydrolase; 5) the amino acid and nucleotide sequences for prolyl endopeptidase; 6) the amino acid and nucleotide sequences for peroxisomal long-chain acyl-CoA thioesterase; 7) the amino acid and nucleotide sequences for an arylacetamide deacetylase-like polypeptide; 8) the amino acid and nucleotide sequences for an epoxide hydrolase-like polypeptide; 9) the amino acid and nucleotide sequences for an epoxide hydrolase-like polypeptide; 10) the partial amino acid and nucleotide sequences of lysophospholipase-1. Numbers to left of sequences indicate amino acid or nucleotide position. GenBank Accession numbers are shown. “X” indicates amino acid residue not known. “n” indicates nucleotide not known.

FIG. 3 is a flow chart for making an activity based probe (see Example 2).

### DETAILED DESCRIPTION OF THE INVENTION

Compositions and methods are provided that can identify neoplastic epithelial cells by differences in the profile of serine-threonine hydrolases, and that can monitor the response of the cells to changes in the environment to which the cell is exposed. As disclosed herein, various serine-threonine hydrolases differ in their level of activity in normal cells as compared to neoplastic cells. Examination of the identified proteins can contribute to an understanding of neoplastic processes; allows for an identification of specific cells and cell types, including normal cells and neoplastic cells; allow a determination of the response of a cell to changes in the environment; and provides targets for the treatment of neoplasia. For example, as disclosed herein, examination of the activity of prostate specific antigen, PSA, can be used to monitor prostate cancer.

The enzymes disclosed herein as useful for monitoring the presence or progression or a disease state, or for selecting a therapeutic intervention or likely efficacy of a selected therapy, include enzymes that are found in both the soluble and insoluble fractions of a cell, including from a cell lysate, from neoplastic prostate epithelial cells. The regulation of these two general classes of proteins in normal cells as compared to neoplastic cells is substantially different, though a few of the same proteins found in the two fractions. Generally, however, fractionation of cells into soluble and insoluble fractions results in substantially different compositions of the enzymes of interest.

The present invention provides isolated polypeptides, including isolated proteins such as an isolated dipeptidyl peptidase (DPP) having an apparent molecular mass of about 70 kDa to 95 kDa, isolated protein conjugates comprising an active enzyme and a probe as defined herein, and isolated antibodies, which are specific for a protein or protein conjugate as disclosed herein. As used herein, the term “isolated” or “purified” refers to a molecule such as a polypeptide, nucleic acid molecule, or the like, that is in an environment other than the environment in which the molecule is normally found in nature. In general, an isolated polypeptide such as a purified enzyme or antibody contains at least about 10% by weight (weight %) of protein of the desired product, generally at least about 25 weight % of protein, usually at least about 50 weight %, and particularly at least about 90 weight %. Desirably, the isolated molecule contains less than about 1 weight % of any other chemically similar molecule, for example, an isolated antibody having a desired specificity contains less than about 1% weight % of any other proteins, including any other antibodies.

Prostate cancer (PCa) is the most commonly diagnosed form of cancer in men in the United States. There are multiple stages that define prostate cancer; they range from benign prostatic hyperplasia (BPH) to prostatic intraepithelial neoplasia (PIN) to metastatic disease. Although PCa is characterized by the transitions through these stages, the disease is slowly progressing and is generally considered a cancer of the aged. It is this slow progression that often makes the disease difficult to diagnose as it is often detected at later stages. One of the most serious hallmarks in the progression of PCa is the transition of the tumor from being hormone sensitive to hormone refractory. This is a key issue as one of the treatments in the early stages of prostate cancer is androgen ablation therapy. Because there are multiple stages that define the status of PCa, one of the ways to diagnose prostate cancer and determine the course of therapy relies on biomarkers, genes or proteins associated with prostate cancer.

Relevant references relating to prostate cancer include Pizer et al. (The Prostate 2001, 47:102-10) describe fatty acid synthase as a potential therapeutic target in androgen dependent prostrate cancer progression (see, also, Kuhjada, Nutrition 2000, 16:202-8). Dipeptidyl peptidase IV (DPP IV; also referred to as CD26) and cognate compounds have been reported to be associated with prostate neoplastic cells (Gonzalez-Gronow et al., Biochem. J. 2001, 355:397-407; Bogenrieder et al., Prostate 1997, 33:225-32; Vanhoof et al., Eur. J. Clin. Chem. Clin. Biochem. 1992, 30:333-8; Wilson et al., J. Androl. 2000, 21:220-6). A variant form of DPP IV, referred to as DPP IV-β, was reported by Jacotot et al. (Eur. J. Biochem. 1996, 239:248-58; Blanco et al., Adv. Exp. Med. Biol. 1997, 421:193-9).

The most commonly used biomarker in the diagnosis of PCa is prostate specific antigen (PSA), which is a serine proteinase that is expressed in the prostate and the plasma level of which is used as an indicator to stage the progression of the disease. While the free form of PSA is most often used as the indicator, the ratio of free PSA to either of its cognate inhibitors, I-1 proteinase inhibitor or I-2-macroglobulin, are also being assessed for their ability to predict outcome and stage disease. As a move is made into the post genomic era, there have been a number of attempts to identify more or better biomarkers for prostate cancer. Several groups have used cDNA microarrays to identify genes that are differentially expressed at various stages of prostate cancer or in prostate cancer cell lines. In addition, several studies have addressed the change in gene expression associated with androgen treatment. These studies have identified a number of genes not previously associated with prostate cancer. For the most part, however, the biological role of these proteins in PCa has not been investigated. Another caveat to these studies is that they do not address actual protein expression of these genes. To address protein levels, several groups have begun to profile the proteomics of prostate cancer. Unlike gene arrays, proteomics can detect changes in protein expression as well as post-translational modifications such as phosphorylation or glycosylation. Similar to the gene profiling studies though, there is little information regarding the biological roles of the proteins associated with prostate cancer. Because there is still little known about the biology of prostate cancer biomarkers, it remains important to identify the proteins associated with prostate cancer.

The investigation disclosed herein focussed on the serine-threonine hydrolases, which comprises a large and diverse family both structurally and functionally. Because of their diversity in structure and function, the proteins are involved in a wide range of biological activities associated with various biological and pathological conditions including blood coagulation, lipid metabolism, pain sensation and tumor progression. As a whole, these hydrolases are one of the most diverse in terms of enzymatic activity. The catalytic properties of these enzymes range from proteolytic cleavage of peptide bonds to synthesis of fatty acyl chains. Because of their wide ranging enzymatic properties and the roles in so many pathological conditions, serine hydrolases have long been targeted for therapeutic intervention. Accordingly, knowing the gene expression profile or even the protein expression profile of these genes is not sufficient, as it is the enzymatic activity of the proteins that is being targeted for drug development. Recently a method was described to profile serine hydrolase activity in biological samples using fluorophosphonate probes (Liu et al., Proc. Natl. Acad. Sci., USA 96(26):14695-14699, 1999, which is incorporated herein by reference). As disclosed herein, such probes were used to identify an aggregate profile of serine-threonine hydrolase activity in prostate cancer cell lines, and to profile changes in hydrolase activity in response to androgen treatment.

A number of cell lines serve as models for prostate cancer, either in tissue culture or as xenographs. Three of the most common are the LNCaP, DU-145 and PC-3 cell lines. These cell lines exhibit quite different phenotypes when injected into mouse prostates as xenographs. The LNCaP cells are the least invasive while the PC-3 cells are the most invasive. In addition, the LNCaP cell line responds to androgen treatment, while the other two cell lines are hormone refractory. Because of the differences between the cell lines, they were used as a model system to understand the serine hydrolase activity profile of prostate cancer.

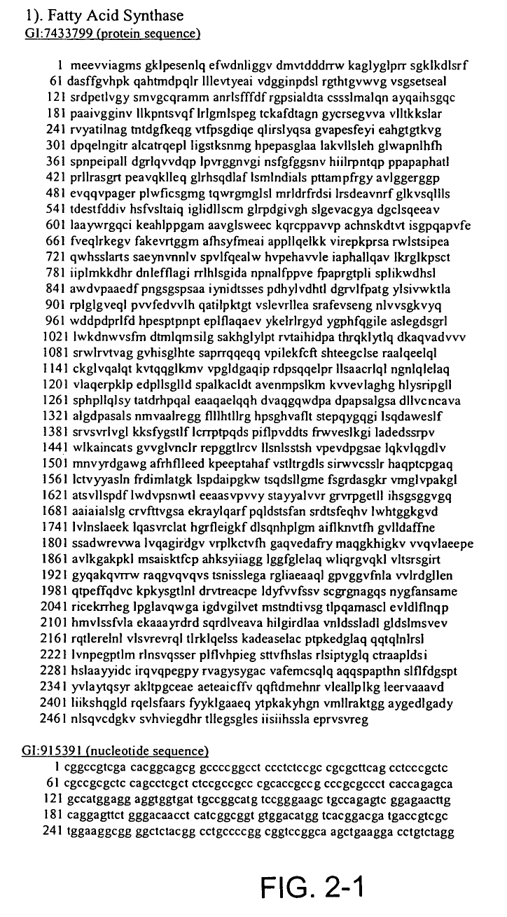

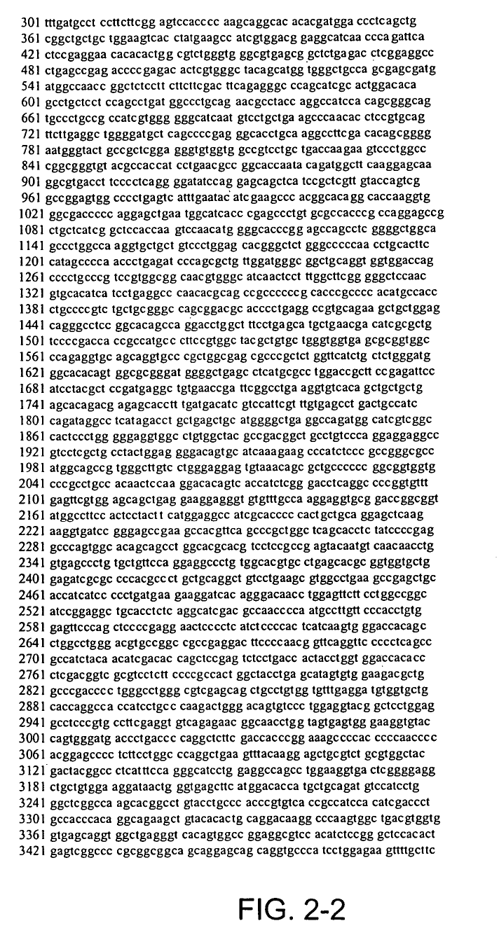

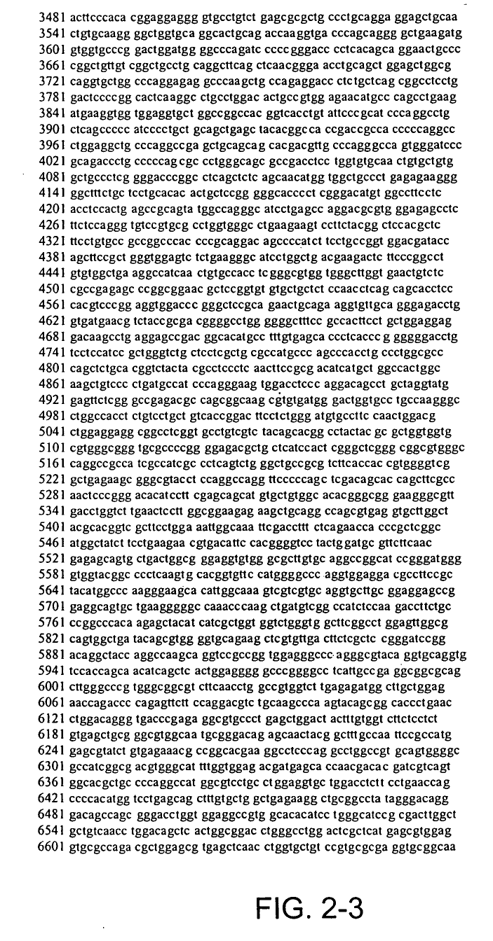

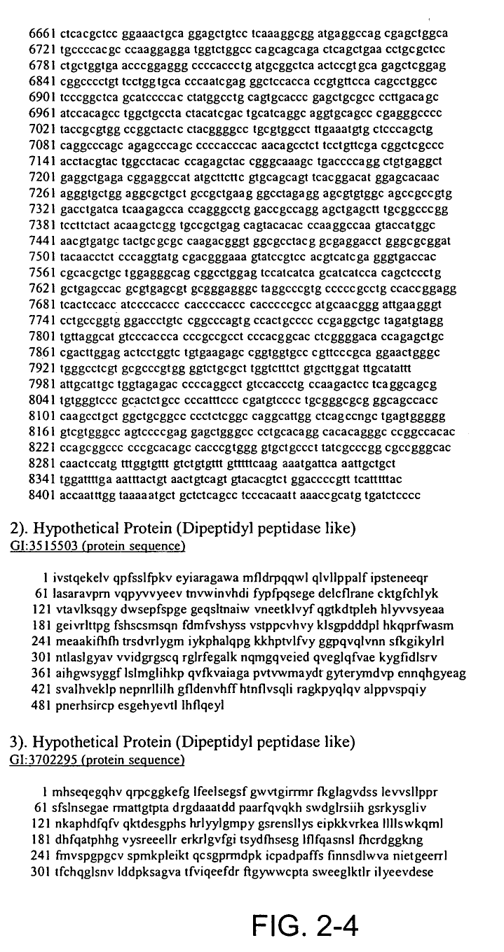

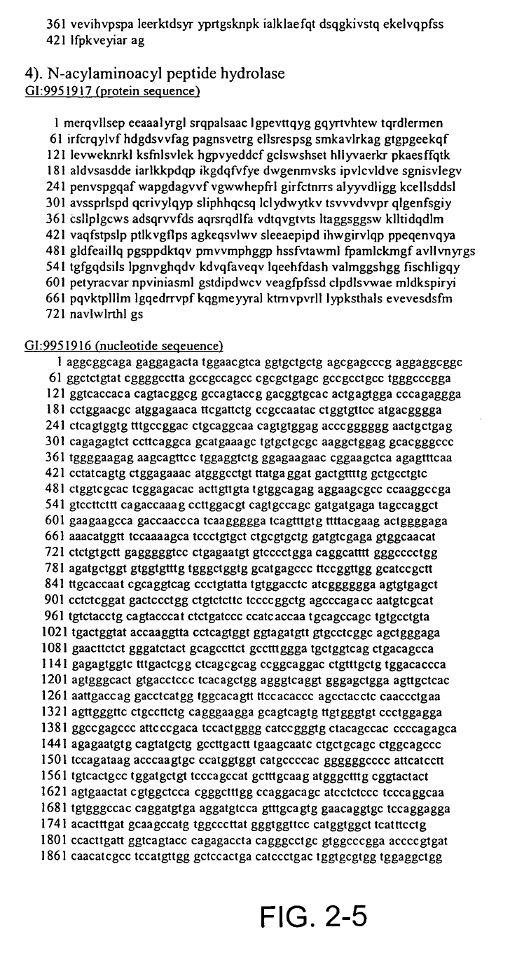

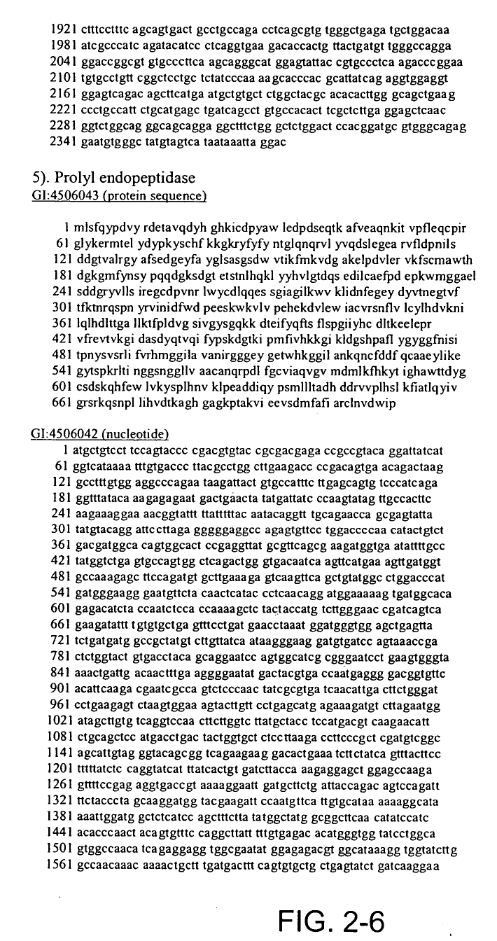

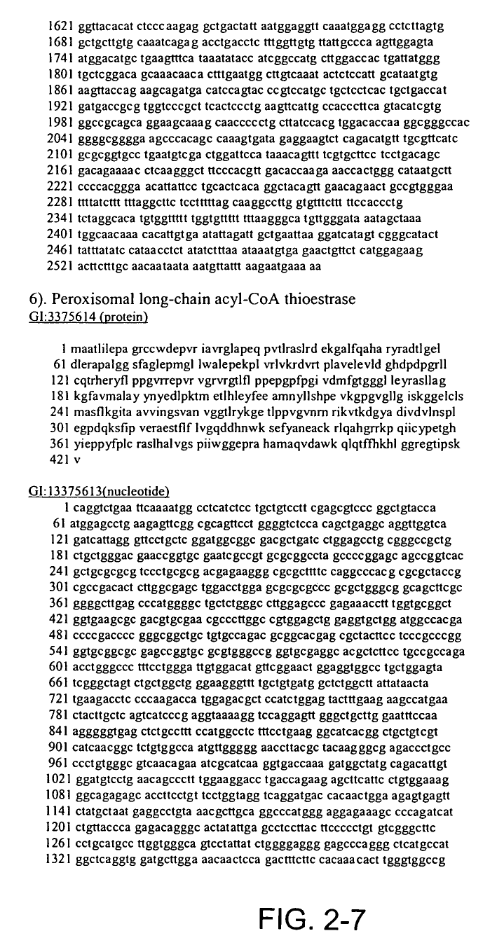

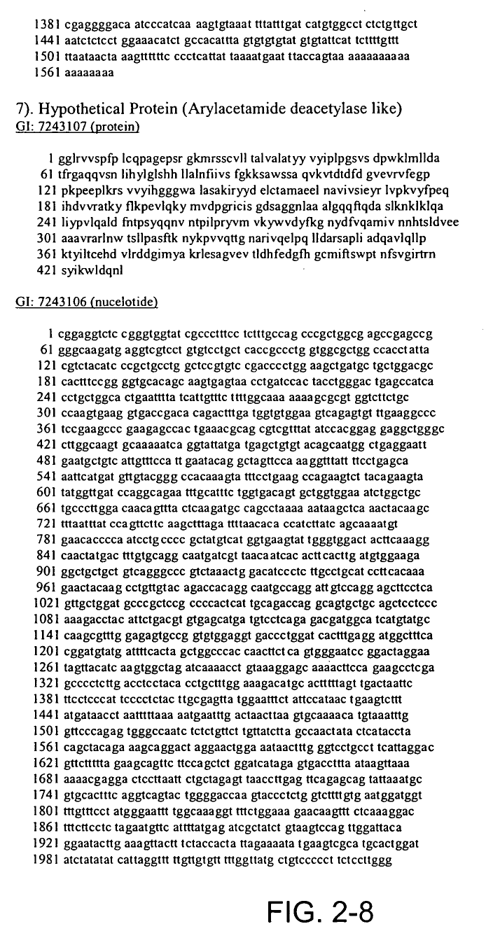

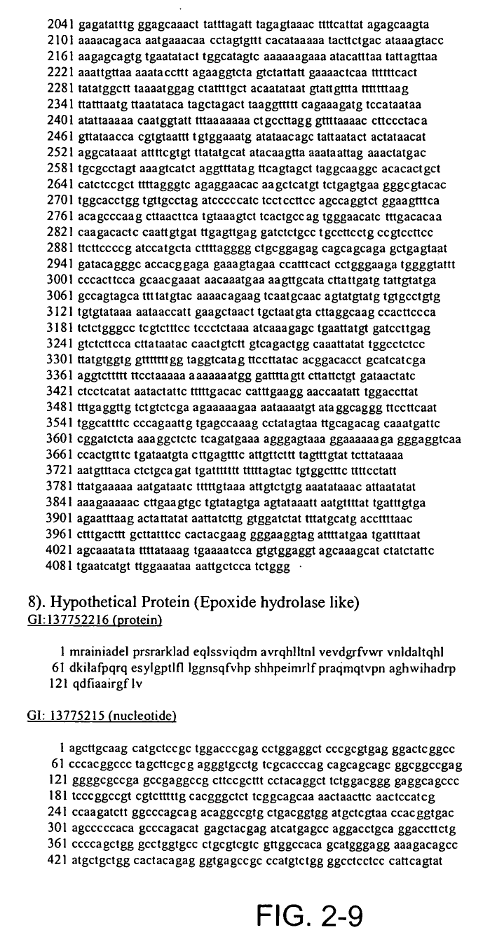

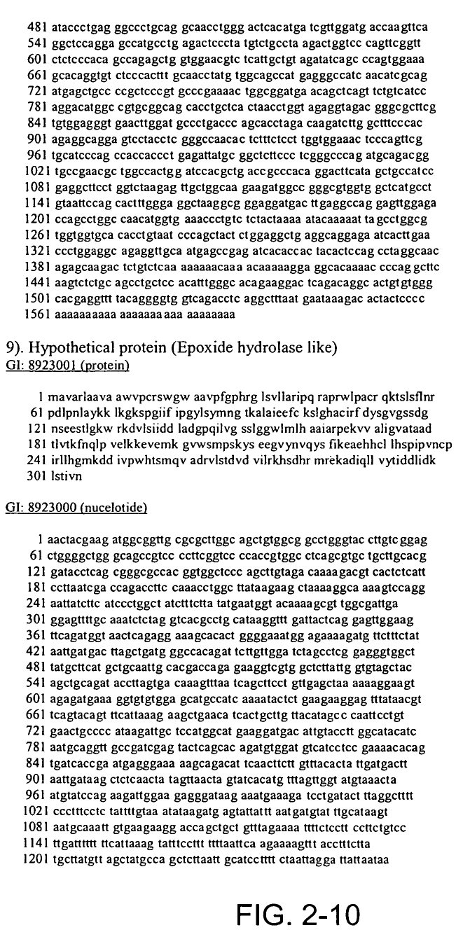

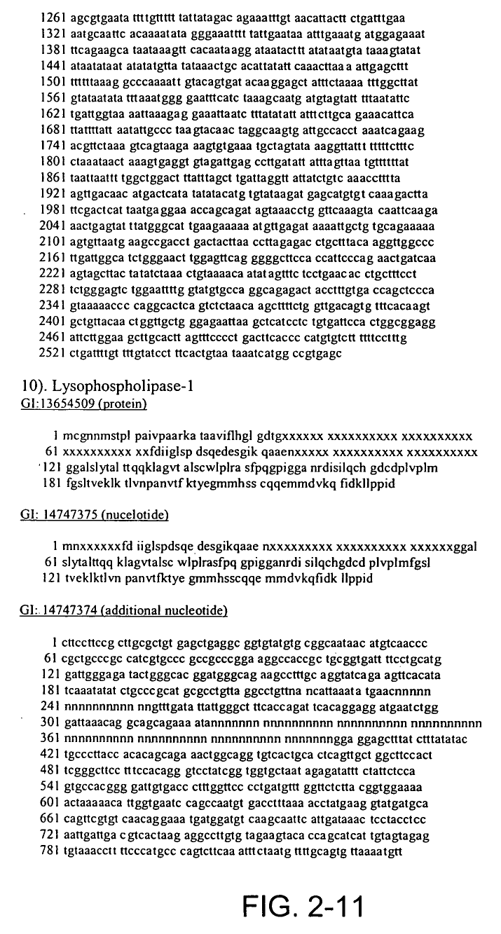

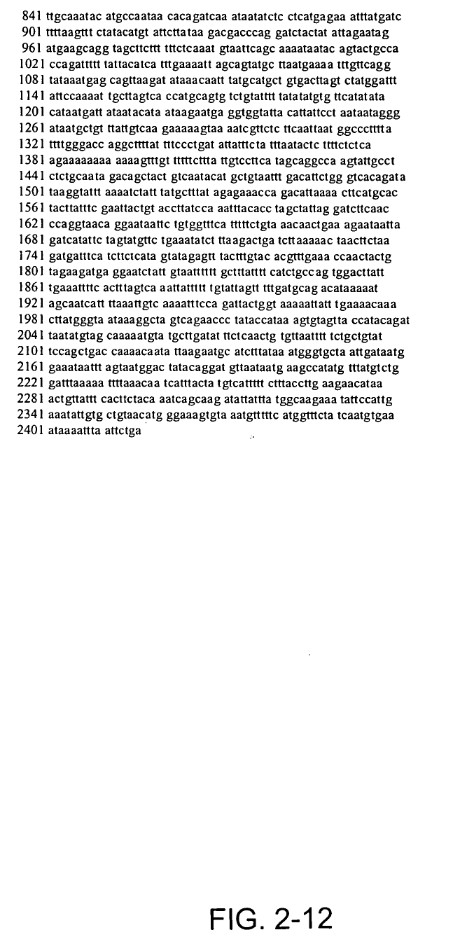

As disclosed herein, the aggregate activity profiles of the three prostate cancer cell lines were quite similar overall. The enzymes that score the highest in terms of activity, as judged by fluorescent labeling, were common to all three cell types (see Example 1), although, within this subset of proteins, the activity levels varied. A scan of the insoluble activity profile identified a large number of proteins that are likely membrane associated, and may be directly responsible for the phenotypic variations between these cell lines. Among the most prevalent enzymes in the three cell lines were five proteins with quite distinct catalytic properties, as expected from the diverse nature of the serine-threonine hydrolase family, including fatty acid synthase, N-acyl peptide hydrolase, prolyl endopeptidase (PEP), long chain coA thioesterase and lysopholipase 1 (see FIG. 2). Only three of these five proteins were also found at detectable levels in normal prostate epithelial PrEC cells though, including PEP, long chain coA thioesterase, and lysophospholipase. PEP, which is the best characterized in terms of biological activity, is an endopeptidase involved in prohormone and neuropeptide processing, though it does not appear to recognize full length proteins. Though PEP is widely expressed, the present disclosure provides the first indication that it is expressed in prostate cells. Long chain CoA thioesterase is required for the biosynthesis and catabolism of fatty acyl chains. This enzyme also is widely expressed, and is likely involved in plasma membrane maintenance. The third protein common to normal PrEC cells and the three cancer cell lines was lysophospholipase 1, which is a recently discovered member of the lipase family. The activity profiles of the four cell lines demonstrated that there are several classes of enzyme activities unique to the cancer cell lines. Included among these are three DPP homologs, which were identified using MS/MS.

The activity profiles of the four cell lines, including the normal cells and the three cancer cell lines, demonstrated that there are several classes of enzyme activities unique to the cancer cell lines. Included among these are three DPP homologs, which were identified using MS/MS sequencing of tryptic peptides. Each of the three cancer cell lines exhibited at least one band associated with the newly described DPP-like activity. The DPP-like proteins do not appear to be closely related in sequence. However, when lysates from each of the three cell lines were preincubated with isoleucine thiazolidide, a known inhibitor of DPP activity, reaction with the fluorescent probe was dramatically reduced (Example 1). A computerized algorithm to search for structural similarity by folding identified the proteins as being related to other DPP family members. No other enzyme activities were inhibited by the treatment. This is the first time the expression of these proteins has been detected, and it is the first functional identification of these proteins as DPP-like enzymes. The present results establish these peptidases as potential contributors to the different phenotypes of these cell lines.

The activity profiles of the four cell lines also demonstrated two other enzyme activities unique to the cancer cell lines, N-acyl peptide hydrolase and fatty acid synthase. N-acyl peptide hydrolase is expressed as a tetramer protein and catalyzes the removal of N-terminal blocked peptides, generating peptides one amino acid shorter than the original substrate. The enzyme does not cleave N-terminally blocked proteins. Despite its wide tissue distribution, the biological function of the enzyme remains unknown. N-acyl peptide hydrolase was absent in small cell lung carcinoma cell lines, where the region of chromosome that encodes the protein is deleted. Although it has been hypothesized that the non-processed N-terminally blocked peptides in these cells are responsible for proliferation of these cells, such a role is not consistent with the enzyme being present in all three prostate cancer cell lines, including the highly aggressive PC-3 cell line, as disclosed herein.

Fatty acid synthase activity was not expressed in the normal PrEC cell line, but was quite active in the prostate cancer cell lines. Fatty acid synthase (FAS) is expressed as a dimer of greater than 500 kDa that catalyzes the formation of fat from other energy sources. For the most part, its expression is limited to tumors and cancer cell lines, as normal tissue utilizes dietary lipids for normal homeostasis. Furthermore, in the case of prostate cancer, FAS expression was associated with aggressiveness. As such, FAS provides a target for therapeutic intervention, and led to the finding that the fungal derived antibiotic cerulenin, and its synthetic derivative C75, are cytotoxic to cancer cells and in some cases induce apoptosis. In the activity profiles disclosed herein, FAS was found in all three prostate cancer cell lines, thus supporting the notion that FAS expression is regulated by several pathways as only one of the three cell lines in this study, LNCaP, responds to androgen.

The transition from an androgen responsive state to an androgen refractory state by a tumor is a major hallmark in the progression of prostate cancer. In the early stages of tumor development, androgen ablation therapy is used to control progression. Once the tumor becomes androgen refractory, ablation therapy becomes useless. Because prostate cancer progresses through different stages of androgen regulation it requires that the proteins and pathways that are active at each stage be identified. Toward that end, the serine hydrolase activity profile of LNCaP cells was profiled in response to DHT treatment (Example 1). Regardless of whether high or low DHT levels are used to treat the cells, the aggregate activity profile changed. This result demonstrate the ability of this system to quantitatively measure changes in enzyme activity in prostate cancer cells, and further identifies these proteins as being hormonal regulated, either directly or indirectly. Moreover, the results indicate that several of the enzymes, including PEP, NAPH and FAS, undergo a post-translational regulation, as the mRNA levels of these enzymes do not change in concordance with activity levels.

The post-translational regulation of enzyme activity is well established, particularly with respect to proteinases. In the case of PEP and NAPH, this level of regulation has not been established and, therefore, it is not clear what modification is being made to these enzymes. In the case of FAS, it has been hypothesized that phosphorylation of the enzyme regulates its catalytic functions, which could explain why the increase in FAS activity is greater that than the increase in mRNA level when LNCaP cells were treated with 0.1 nM DHT (Example 1). The results disclosed herein indicate that a matrix, or combinations, of activity changes are associated with changes in cell dynamics associated with androgen responsiveness.

The enzymes of interest in the soluble fraction comprise a number of categories. In addition to the known dipeptidyl peptidases, DPP-UV and DPP-IV-β, which are associated with up-regulation in prostate hyperplasia, two additional DPPs were identified. The two additional DPPs had molecular weights of about 70 kDa to 95 kDa as determined by mass spectrometry, were present in normal prostate epithelial cells and in prostate cancer cell lines, and were up-regulated in neoplastic cells. Tryptic digests of the DPPs were examined by MALDI-TOF and MS/MS sequencing, and had sequences there were not found in nucleic acid or protein databases. The DPPs further reacted in a lysate with fluorophosphonate probes, which are specific for serine-threonine hydrolases that are enzymatically active, and were inhibited by isoleucine thiazolidide, which is a known DPP inhibitor. The DPP enzymes are expressed on the cell surface and, therefore, can be conveniently detected without requiring that the cells to be examined be lysed or otherwise degraded. The degree of up-regulation is related to the degree of aggressiveness of the cancerous cells, such that comparison of the levels of one or both of these enzymes with standards, for example, cancerous cells of established aggressiveness, can be prognostic of the outcome of the disease and indicate the nature and severity of the treatment.

Other enzymes of interest in obtaining a profile of prostate epithelial cells are present in normal prostate epithelial cells and reduced or absent in cancerous prostate cells, and has a molecular weight of about 60 kDa. As used herein, the term “molecular weight” or “apparent molecular mass” indicates the size of a protein as determined by a method such as mass spectrometry, gel chromatography, denaturing gel electrophoresis, or any other method known in the art as useful for such a characterization of a polypeptide. N-acylaminoacyl peptide hydrolase having a molecular weight (“m.w.”) of about 73 kDa was found in the neoplastic cells, but not the normal prostate epithelial cells. Other serine-threonine hydrolases that distinguished between neoplastic and normal prostate epithelial cells were found in both the soluble and insoluble fractions of prostate epithelial cells, and include fatty acid synthase (m.w. about 217 kDa; Pizer, et al., supra; Kuhajda, supra), prolyl endopeptidase (m.w. about 81 kDa), peroxisomal long chain acyl-CoA thioesterase (m.w. about 47 kDa; Jones and Gould, Biochem. Biophys. Res. Comm. 2000, 275:233-40); a protein having epoxide hydrolase activity (m.w. about 30 kDa), and lysophospholipase-1 (m.w. about 26 kDa). A number of bands of proteins, which were detected in the insoluble fraction of normal prostate epithelial cells, but not of neoplastic prostate epithelial cells, also were identified, and had molecular weights of about 57 kDa, 56 kDa, and 55 kDa; in addition, one or more neoplastic cells had a band of about 50 kDa that was reduced or absent in the normal cells.

As used herein, the term “reduced or absent”, when referring to a protein, means that the particular protein is either present in a decreased amount in a particular cell as compared to reference cell or not detectable using a particular analytic method. It should be recognized that an amount of a protein can be below a level that is detectable by a particular assay. As such, while the absolute presence or absence of a protein may not be detectable, a change in the level can be determined using the methods of the invention such that, for example, a protein is reduced from a detectable level in a normal cell to an undetectable level in a neoplastic cell, or any other qualitative or quantitative change. In general, a protein is considered to be “reduced or absent” if there is less than about 20% of the amount of a protein in the particular cell as compared to a reference cell, e.g., a neoplastic cell as compared to a normal cell, generally is less than about 10%, and usually less than about 1%, as determined, for example, by gel electrophoresis (see Example 1) and at the same level of detection. In referring to a protein being “present in neoplastic cells”, it is intended that the protein be detectable in at least two different neoplastic prostate epithelial cell lines, for example, any two of the exemplified LNCaP, DU145, and PC3 cell lines.

By virtue of the differences in enzyme activity levels in prostate cells having different neoplastic activity, ranging from non-cancerous to aggressive, screening prostate cells for one or a plurality of serine-threonine hydrolases can be diagnostic of the disease and informative of a course of treatment. The screening is associated with a determination of the level of enzyme activity in the cells, rather than the total amount of enzyme. Of course, by comparing activity level with the total amount of an enzyme in the cells, where the total amount of enzyme is correlated with the activity level, one can use either measure. The expression level and level of active enzyme can be related to the boundary between hyperplasia and neoplasia, the stage of cancerous tissue at the different lobes of the prostate and the diagnosis of cancer based on histology.

The present methods provide a means to identify active proteins, particularly active serine-threonine hydrolase enzymes. The enzymes are identifiable using probes that distinguish between active and non-active enzymes. As used herein, the term “active” refers to an enzyme that is in an enzymatically active conformation and able to catalyze its normal reaction. As such, the enzyme is not substantially denatured, is in a relatively native conformation for receiving substrate, and is not complexed with an inhibitor that prevents access to the active site. A number of probes have been identified that use labeled reactive compounds to react with the active serine-threonine hydrolases that provide different profiles for mixtures of serine-threonine hydrolases. These compounds are referred to as activity-based probes (ABPs), and, where fluorescently labeled, are referred to as fABPs.

The probes can be divided into four general regions: 1) a functional group (F) that specifically and covalently bonds to the active site of a protein; 2) a detectable label or a ligand (collectively “ligand”) for sequestering and/or detecting a conjugate of the ABP and an active protein (X); 3) a linker L, positioned or formed between the F and the L; and 4) a binding moiety or affinity label that can be associated with or part of the linker region and/or the functional group (R). The linker can be a bond or chemical group used to link one moiety to another, serving as a divalent bridge, where it provides a group between two other chemical moieties. A binding or affinity moiety can be any chemical group, including a single atom, that is conjugated to the reactive functional group or associated with the linker, as a side chain or in the chain of the linker, and provides enhanced binding affinity for protein targets. The ligand can be used to detect and/or capture the ABP in combination with any other moieties that are bound strongly to the ligand so as to be retained in the process of the reaction of the functional group with the target active protein. The ABP can include a chemically reactive functionality, not found in proteins, that can react with a reciprocal functionality, e.g., a vic.-diol with boronic acid, an aldehyde, a ketone, etc. Such reactive functionalities can be used to bind to a ligand after reaction with the target protein. The ABP also can be truncated, and lack the ligand, but always contains a functional group (F), a linker (L), and an R group (binding moiety).

An ABP has a fluorophosphonate electrophile, which can have a different environment for mixtures of ABPs, so as to have different target specificities. A single ABP or mixture of ABPs can be used in the methods disclosed herein, and the environments can be different, the labels can be different, or both. An ABP can be illustrated by the formula
R*(F-L)-X

where the symbols are as defined previously, the asterisk indicates that R can be included in F or L, and X is bonded to L; more specifically, wherein,

X is a ligand present prior to formation of a protein conjugate product or added to a reactive functionality to provide the ligand and, where the ABP comprises a member of library of ABPs, the ligand has the same chemical structure for each of the members of the library;

L is a bond or linking group, which is the same in each of the members of a library of ABPs;

F is a functional group reactive at an active site of a protein member, wherein the functional group comprises the same reactive functionality in each of the members of a library of ABPs; and

R is a group having a molecular weight less than about 1 kDa, and is different in each of the members of a library of ABPs; and the * indicates that R is a part of F or L;

and wherein, where the ABP is a member of a library of ABPs, the members of the library have different on rates with the protein member. For example, when X is biotin or any ligand, L is any linker of varied composition and length, F is a sulfonate, and R is a pyridyl group, a distinct protein profile is observed as compared with the same ABP where the R group is methyl. Thus by varying R when bonded to a sulfonyl group, different binding profiles are obtained, and specificity can be identified, thus providing a means to design a drug based on the structure of R or to look for binding to related target proteins for proteome analysis.

The functional group (F—R) reactive with an active protein can be, for example, a sulfonate ester having R as any group such as alkyl, heterocyclic, pyridyl, substituted pyridyl, imidazole, pyrrole, thiophene, furan, azole, oxazole, aziridine, aryl, substituted aryl, amino acid or peptidyl, oligonucleotide, or carbohydrate group. The ligand portion permits capture of the conjugate of the target protein and the probe. The ligand can be displaced from a capture reagent by addition of a displacing ligand, which may be free ligand or a derivative of the ligand, or by changing solvent (e.g., solvent type or pH) or temperature conditions or the linker may be cleaved chemically, enzymatically, thermally or photochemically to release the isolated materials (see discussion of the linker moiety, below). Examples of ligands (X), including labels, include, but are not limited to, biotin, deiminobiotin, dethiobiotin, vicinal diols, such as 1,2-dihydroxyethane and 1,2-dihydroxycyclohexane, digoxigenin, maltose, oligohistidine, glutathione, 2,4-dintrobenzene, phenylarsenat, ssDNA, dsDNA, a peptide or polypeptide, a metal chelate, a saccharide, a fluorescer such as rhodamine or fluorescein, or a hapten to which a specific antibody can be generated. Examples of ligands and their capture reagents include but are not limited to dethiobiotin or structurally modified biotin-based reagents, including deiminobiotin, which bind to proteins of the avidin/streptavidin family, for example, in the form of streptavidin-agarose, oligomeric avidin-agarose, or monomeric avidin-agarose; a 1,2-diol such as 1,2-dihydroxyethane (HO—CH₂—CH₂—OH), and other 1,2-dihyroxyalkanes, including those of cyclic alkanes such as 1,2-dihydroxycyclohexane, which bind to an alkyl or aryl boronic acid or boronic acid ester such as phenyl-B(OH)₂ or hexyl-B(OEthyl)₂, which can be attached via the alkyl or aryl group to a solid support material, such as agarose; maltose, which binds to maltose binding protein (as well as any other sugar/sugar binding protein pair or, more generally, to any ligand/ligand binding protein pairs having the properties discussed above; a hapten such as the dinitrophenyl group, which binds to an anti-hapten antibody, for example, an anti-dinitrophenyl-IgG; a ligand that binds to a transition metal, for example, an oligomeric histidine, which binds Ni(II), wherein the transition metal capture reagent can be in the form of a resin bound chelated transition metal such as nitrilotriacetic acid-chelated Ni(II) or iminodiacetic acid-chelated Ni(II); glutathione which binds to glutathione-S-transferase; and the like.

In general, any affinity label-capture reagent that is commonly used for affinity enrichment and that meets the suitability criteria discussed above can be used to prepare an ABP and, therefore, can be used in a method of the invention. Biotin and biotin-based affinity tags are illustrated herein, including structurally modified biotins such as deiminobiotin or dethiobiotin, which can be eluted from avidin or streptavidin (strept/avidin) columns with biotin or under solvent conditions compatible, for example, with ESI-MS analysis (e.g., in dilute acids containing 10-20% organic solvent). For example, a deiminobiotin tagged compound can be eluted in a solvent having a pH less than about pH 4.

The linker group can be a bond, though generally is other than a bond. For example, the linker group can be a cleavable linker group, which can be cleaved by a thermal, chemical, photochemical or other reaction. The choice of linker, as with the choice of an R group, contributes to the specificity of an ABP. A photocleavable groups in a linker, for example, can include a 1-(2-nitrophenyl)ethyl group. A thermally labile linker can include a double stranded duplex formed from two complementary strands of nucleic acid, a strand of a nucleic acid with a complementary strand of a peptide nucleic acid, or two complementary peptide nucleic acid strands that can dissociate, for example, upon heating. A cleavable linker also can include a linker comprising a disulfide bond, acid or base labile groups such as a diarylmethyl or trimethylarylmethyl group, or a silyl ether, carbamate, oxyester, thioester, thionoester, or alpha-fluorinated amide or esters. An enzymatically cleavable linker can contain a protease-sensitive amide or ester, a θ-lactamase-sensitive θ-lactam analog, or can contain a nuclease-cleavable or glycosidase cleavable bond.

Linker groups include, among others, ethers, polyethers, diamines, ether diamines, polyether diamines, amides, polyamides, polythioethers, disulfides, silyl ethers, alkyl or alkenyl chains (straight chain or branched and portions of which may be cyclic) aryl, diaryl or alkyl-aryl groups. Where an amino acid or oligopeptide is used, it generally comprises an amino acid having 2 to 3 carbon atoms, e.g., glycine and alanine. Aryl groups in linkers can contain one or more heteroatoms (e.g., N, O or S atoms). Linkages also include substituted benzyl ethers, esters, acetals or ketals, diols, and the like (see, U.S. Pat. No. 5,789,172, which is incorporated herein by reference; listing useful functionalities and manners of cleavage). The linkers, when other than a bond, will have from about 1 to 60 atoms, generally about 1 to 30 atoms, where the atoms include C, N, O, S, P, etc., generally C, N and O, and usually have from about 1 to 12 carbon atoms, including about 0 to 8, particularly 0 to 6 heteroatoms. The atoms are exclusive of hydrogen in referring to the number of atoms in a group, unless indicated otherwise.

The linker and/or the ligand can be isotopically labeled, for example by substitution of one or more atoms in the linker with a stable isotope. For example, ¹H can be substituted with ²H or ¹²C can be substituted with ¹³C. Alternatively, one atom can be substituted for another, for example, H can be substituted with F, or unsaturation or other such means can be used to provide a mass difference. While ligands or linking groups can have different isotopic distributions, for the purposes of the present invention they generally are considered to be of the same chemical composition, where the atomic numbers of the atoms and their organization in the ligands or linking groups is the same. Therefore, in one aspect, the method of the invention provides for labeling of the ligand and/or linker to facilitate quantitative analysis by mass spectrometry of the amounts of active proteins in different samples or in samples subjected to different conditions, for example, in the presence and absence of a drug. The label or linker also can be non-radioisotopically labeled, for example, with a fluorophore. In one aspect, the label produces an electromagnetic signal.

The process and compositions described in WO 00/11208, which is incorporated herein by reference, can be used with respect to the present invention. In such an application, an affinity tagged, substantially chemically identical and differentially isotopically labeled probe is used, and the conjugates or fragments thereof are identified by mass spectrometry. The ratio of the different isotopic probes for each of the proteins with which the probes have reacted provides for the relative quantities of the individual proteins.

Linkers can vary widely and can include alkyleneoxy and polyalkyleneoxy groups, where alkylene is of from 2 to 3 carbon atoms, methylene and polymethylene, polyamide, polyester, and the like, where individual monomers generally comprise about 1 to 6 carbon atoms, usually 1 to 4 carbon atoms. The oligomers generally have about 1 to 10 monomeric units, usually 1 to 8 monomeric units, which can be, for example, amino acids, either naturally occurring or synthetic; oligonucleotides, either naturally occurring or synthetic; condensation polymer monomeric units; or combinations thereof. Alteration in the linker region alters the specificity of the ABP for a target protein or class of proteins (e.g., enzymes).

An advantage of initially examining a proteome with a library of ABPs is that one or a few probes can be identified that are specific for target proteins and provide information about the active site of the protein or related group of proteins. Upon identifying such a probe or probes, for example, by mass spectrometry, fluorometry, or electrochemically, or a combination of such detection methods, the one or few probes then can be used singly or in combination in a proteome mixture. The target proteins or proteins then can then be determined using conventional methods such as immunoassays, if available, sequencing, mass spectrometry, and the like. The particular affinity label or labels also can provide a basis for the design of a drug that is specific for the target protein.

Screening assays such as FACS sorting and cell lawn assays can be used to detect the ABP. When ligand (X) is detached prior to evaluation, its relationship to a solid support can be maintained, for example, by location within a grid of a standard 96 well plate or by location of activity on a lawn of cells. Regardless of whether the compounds are tested attached to or detached from a solid support, tags attached to the solid support that are associated with bioactivity can be decoded to reveal the structural or synthetic history of the active compound (see for example, Ohlmeyer et al., Proc. Natl. Acad. Sci., USA 90, 10922-10926, 1993). The usefulness of such libraries as screening tools was demonstrated by Burbaum et al. (Proc. Natl. Acad. Sci., USA 92, 6027-6031, 1995).

The use of a ligand comprising a fluorophore (hereinafter “fluorescer”) provides the advantage that it can be excited when in a gel and the emitted light desirably used to quantitate the amount of fluorescer and, therefore, the amount of protein, present in the excitation light pathway. As discussed above, the ligand also can be a small molecule, for example, a small binding molecule that binds a naturally occurring receptor, or a hapten for which a specific antibody is available. Such an antibody can be raised by binding the hapten to a carrier molecule such as bovine serum albumin or keyhole limpet hemocyanin, thus providing an immunogen that can be used to immunize a mammalian host. The resulting antiserum can be purified and made specific for the hapten, or B lymphocytes of the immunized host can be used to produce hybridomas, which are immortalized cells that produce monoclonal antibodies specific for the hapten. Among natural ligands and receptors are biotin and strept/avidin or analogs of biotin, e.g. dethiobiotin and deiminobiotin, sugars and lectins, substrates and enzymes, and the like. The ligands find particular use for sequestering the reaction product of the probe and target, which then can be fractionated into individual products and analyzed. By having the receptor bound to a surface or other solid support such as a bead, a vessel wall, a glass or silicon slide, or the like, all of the reaction products can be sequestered followed by release and analysis. As discussed below, the probe can have both a ligand and a fluorescer. Where there is no fluorescer present, fractions to be separated can be contacted with a labeled receptor, which can bind to and allow visualization of the product.

The fluorescers can be varied widely depending upon the protocol to be used, the number of different probes employed in the same assay, whether a single or plurality of lanes are used for a gel electrophoresis procedure, the availability of excitation and detection devices, and the like. Particularly useful fluorescers absorb light in the ultraviolet or visible range and emit light in the ultraviolet or visible range, particularly emission in the visible range. Absorption generally is in the range of about 250 nm to 750 nm and emission generally is in the range of about 350 nm to 800 nm. Illustrative fluorophores include xanthene dyes; naphthylamine dyes; coumarins; cyanine dyes; and metal chelate dyes such as fluorescein, rhodamine, rosamine, BODIPY, dansyl, lanthanide cryptates; erbium, terbium and ruthenium chelates, for example, squarates, and the like. The literature amply describes methods for linking the fluorescers through a wide variety of functional groups to other groups (see, for example, Hermanson, “Bioconjugate Techniques” (Academic Press 1996)). The fluorescers have functional groups that can be used as sites for linking, and generally have a molecular weight less than about 2 kDa, usually less than about 1 kDa.

Matched dyes also can be useful for practicing the methods of the invention (see U.S. Pat. No. 6,127,134; describing labeling proteins with dyes that have different emissions, but have the same migratory aptitude in electrophoresis). The term “same migratory aptitude” is used herein to indicate that dyes, when bound to the same molecule (e.g., a protein), at the same site, and in the same way, form conjugates that form a substantially superimposable band upon being subjected to gel electrophoresis. The cyanine dyes can be particularly useful for this purpose because of their positive charge, which matches the charge of lysine, to which cyanine dyes bind. In addition there is the opportunity to vary the polyene linker, while keeping the molecular weight about the same with the introduction of an alkyl group in the shorter polyene chain dye to offset the longer polyene. Also described are the BODIPY dyes, which lack a charge. The advantage of having two dyes that similarly affect the migration of the protein would be present when comparing the native and inactivated samples, though such a procedure also requires that, in the inactivated sample, at least a portion of the protein is monosubstituted.

It also can be desirable to have a ligand bound to a fluorescent ABP (fABP) such that all of the fABPs, conjugated or unconjugated, can be captured and washed free of other components of the reaction mixture. This can be of particular interest where the protein bound to the fABP is partially degraded, leaving an oligopeptide that is specific for the protein and can be analyzed with a mass spectrometer. Also, the ligand allows for a cleaner sample to be used for electrophoretic separation by capture, wash and release. The ligand is generally less than about 1 kDa, and biotin is a conventional and convenient ligand, particularly biotin analogs such as dethiobiotin and deiminobiotin, which can be readily displaced from strept/avidin by biotin. However, any small molecule will suffice, provided it can be captured and released under convenient conditions. The ligand is placed distant from the functional group, generally by a chain of at least about 3 atoms, usually at least about 4 atoms.

Having identified the proteins having different levels of activity between the different prostate epithelial cells, e.g., stages of neoplastic cells and normal cells, cells from patients, including cells obtained by a biopsy procedure, cells sloughed into the blood stream, and the like, can be screened. The cells can be processed prior to analysis, depending on the manner in which they are isolated. A tissue sample, for example, can be treated to separate the cells from matrix components, then the isolated cells used directly in an assay or can be expanded using routine methods. Cells can be isolated from blood using panning, a FACS technique, a centrifugation step, or any other convenient and routine separation technique. The cells can be further washed and harvested, then lysed by any convenient conventional means, including, for example, sonication, mechanical disruption, or osmotic pressure, provided that the methods used do not denature the target proteins (enzymes), which retain their activity. Additives can be included in the lysate, for stabilization, oxidation prevention, pH maintenance, and the like. Various conventional buffers can be employed, consistent with the assay, such as Tris, PBS, MOPS, etc., where the pH generally is in the range of about pH 6.5 to 9, particularly about pH 7 to 8.

In one embodiment, a cell lysate is fractionated into soluble and insoluble fractions, either or both of which can be assayed according to a method of the invention. Such fractionation can be readily achieved by centrifugation, filtration, or any other convenient method. The insoluble fraction can be further dispersed in a medium, conveniently the same buffer used for the preparation of the lysate, and the protein concentration can then be adjusted, for example, where a semi-quantitative or quantitative determination is desired. Optionally, a known amount of a known protein, which is not otherwise present in the sample or is present in a known amount, can be added to the reactants to normalize the amounts of the proteins of interest being examined within or among a number of assays.

The assay can be performed as a single assay or in replicates, and can include one or more standards, controls, and the like. A standard, for example, can be a normal cell, which can be a primary cell, a cell of one or more known cell lines having known protein profiles, or primary neoplastic cells, which can be from a source other than the sample to be assayed. A control, for example, can lack any protein. Where the ABP is labeled with a ligand that allows isolation of the reaction product of the protein and the ABP, the lysate can be treated with the receptor for the ligand, so as to remove any endogenous ligand that is present. For example, if the ligand is biotin, then the lysate can be treated with streptavidin, which can be bound to a solid support or other entity that allows for ready separation, to remove endogenous biotin.

A solution containing the proteins from the sample is then mixed with one or more of the ABPs, which can be in the same or different samples. If in the same sample, each of the ABPs is distinctively labeled such that each is separately detectable. For the most part, different ABPs will be used in different vessels, so as to be able to act independently. Mild reaction conditions are employed, generally a temperature in the range of about 10° C. to 40° C.; the amount of total protein in the sample generally is about 0.05 mg/ml to 5 mg/ml, usually about 0.5 mg/ml to 2 mg/ml; and the amount of ABP generally is in the range of about 0.1 TM to 10 μM, usually about 1 TM to 5 μM. The mixture is incubated for a sufficient time such that the reaction can proceed to at least about 60% completion, generally at least about 80% completion, and particularly to substantially 100% completion. Alternatively, measurements can be made kinetically, wherein samples, which can be duplicate or more, are taken at fixed times, generally at least two different times. Depending on the probe and concentrations of the components of the assay medium, the reaction generally will be allowed to proceed for at least about 10 minutes, and usually not more than about 6 hours (though it can be allowed to proceed overnight if convenient), and more usually is allowed to proceed for at least about 30 minutes and not more than about 3 hours.

The analysis of the data will vary depending on the information desired from the assay. For example, if the amount of individual protein complexes is to be determined, and if the migration rate of the complexes is known, an electrophoresis procedure, for example, slab, capillary or microfluidic electrophoresis, can be used to separate the components. The fluorescence of each band can be determined, and is indicative of the amount of active protein target in the sample. A method such as HPLC or other chromatographic technique, which provides for separation of the proteins into individual fractions, also can be used. For further characterization, the western blot analysis can be performed. In addition, the complexes can be extracted from the gel, digested with a protease or other proteolytic agent, and the digestion fragments analyzed, for example, by mass spectrometry.

Where the total amount of available target protein is equivalent to or can be correlated with the amount of an active target protein, the proteins can be further analyzed using other methods than a method using ABPs. For example, an immunoassay can be employed, wherein the antibodies bind to the target protein in a competitive or non-competitive manner, or any other convenient assay can be used. The assay format can be any format, including, for example, an ELISA, EMIT, SLFIA, CEDIA, or FRET assay.

The identified active proteins can form a profile that is the basis of diagnostic assay for determining, for example, whether metastatic cells are prostate cells. The protein profile also be used, for example, to follow the response of prostate cancer cells to a treatment such as brachytherapy, radiation therapy, chemotherapy, hormone therapy or other therapy used for the treating prostate cancer. A biopsy can be taken using routine clinical methods, and the cells obtained can be analyzed for the proteins and protein profile in order to determine the extent to which the cancerous cells have been ablated, wherein changes in the levels of the different active proteins are related to the response to the treatment. Increases and decreases in the amount of activity of one or more of the proteins can be monitored during the course of the treatment along with other indicia of presence of cancerous epithelial cells such as PSA and PSCA levels, thereby greatly enhancing the level of confidence as to the efficacy of a treatment.

The identified active proteins can be used as reagents in screening assays to identify compounds having a desired binding affinity for the protein. By employing a competitive assay between the ABP and the compound being screened, and allowing the reaction to proceed with only partial bonding of the probe to the protein, changes in the amount of bonding of the probe over a predetermined time indicates the affinity of the compound for the protein. Of course, reagents can be used other than the ABPs that compete for the active site to determine binding affinity, including, for example, a substrate or substrate analog for the active protein, where the protein is an enzyme. The neoplastic cells can also be used in a screening assay, wherein the amount of the active protein formed in the presence and absence of the compound is determined, using the ABP.

Preparation of antibodies, including antisera, polyclonal antibodies, and monoclonal antibodies, can be according to routine methods. Polyclonal antibodies generally are raised in animals by multiple subcutaneous, intradermal, or intraperitoneal injections of the protein and an adjuvant. In some cases, it can be useful to conjugate the protein or a peptide fragment of the protein containing the target amino acid sequence, to a carrier molecule that is immunogenic in the species to be immunized, for example, a carrier molecule such as keyhole limpet hemocyanin, serum albumin, bovine thyroglobulin, or soybean trypsin inhibitor. The conjugation can be performed using a bifunctional or derivatizing agent, for example, maleimidobenzoyl sulfosuccinimide ester (conjugation through Cys residues), N-hydroxysuccinimide (through Lys residues), glutaraldehyde, succinic anhydride, SOCl₂, dialkyl or cycloalkyl carbodiimide.

Host animals can be immunized by combining 1 mg or 1 μg of conjugate (for rabbits or mice, respectively) with 3 volumes of Freund's complete adjuvant, and injecting the solution intradermally at multiple sites. One month later the animals are boosted with 1/5 to 1/10 the original amount of conjugate in Freund's complete adjuvant by subcutaneous injection at multiple sites. Seven to 14 days later, the animals are bled and the serum is assayed for antibody titer. Animals are boosted until the titer reaches a plateau. Preferably, the animal is boosted with the same protein or peptide fragment, but conjugated to a different protein or through a different cross-linking agent. Conjugates also can be made in recombinant cell culture as protein fusions. Also, aggregating agents such as alum can be used to enhance the immune response.

Monoclonal antibodies are prepared by recovering spleen cells from immunized animals and immortalizing the cells in conventional fashion, for example, by fusion with myeloma cells to produce hybridomas, or by Epstein-Barr virus transformation and screening for clones expressing the desired antibody. B lymphocytes can be obtained by removing the spleen or lymph nodes of sensitized animals in a sterile fashion and carrying out a cell fusion to produce hybridoma cells. Alternately, lymphocytes can be stimulated or immunized in vitro (see, for example, Reading, J. Immunol. Meth., 53:261-291, 1982). A number of cell lines suitable for cell fusion have been developed, and the choice of any particular cell line for hybridization protocols in the production of monoclonal antibodies is directed by any one of a number of criteria such as speed, uniformity of growth characteristics, deficiency of its metabolism for a component of the growth medium, and potential for good fusion frequency.

Successfully fused hybridoma cells can be separated from the parental B lymphocytes and myeloma cell line using any convenient methods, for example, by incubating the cells in a selective medium such hypoxanthine-aminopterin-thymidine (HAT) medium, wherein only the hybridoma cells can survive and proliferate. Surviving hybridoma cells are subjected to limiting dilution, and antibodies that are produced by cloned hybridoma cell line and having the desired specificity are identified, for example, by contacting medium from the hybridoma cultures with the antigen, which generally is immobilized to a solid support such as a plastic well of a 96 well plate, and identifying specific binding. Hybridoma cells producing the desired antibody then can be grown in larger cultures, as desired, and aliquots can be stored, for example, in liquid nitrogen, thereby providing a convenient and long term source of the desired monoclonal antibodies.

Where it is desired to obtain higher concentrations of the antibodies, hybridoma cells can be transferred into animals to obtain inflammatory ascites, and antibody-containing ascites fluid can be collected 8 to 12 days later. The ascites fluid contains a high concentration of antibodies, but includes both the monoclonal antibodies and immunoglobulins generated in response to the inflammatory ascites. Antibody purification can be achieved, for example, by affinity chromatography (see Harlow and Lane, “Antibodies: A Laboratory Manual” (Cold Spring Harbor Laboratory Press 1998; Harlow and Lane, “Using Antibodies: A Laboratory Manual” (Cold Spring Harbor Laboratory Press 1998).

For therapeutic antibodies, the antibodies will generally be “human” or humanized antibodies. Humanized and “human” antibodies are described in U.S. Pat. Nos. 6,235,883; 6,254,868; and 6,258,562, and can be obtained from commercial sources (see, for example, Abgenix, Inc.; Fremont Calif.). The use of antibodies and such conjugates is described in U.S. Pat. Nos. 5,441,871; 5,443,953; 6,071,519; 6,077,519; 6,103,235; 6,160,099; 6,196,299; 6,214,388; 6,214,973; 6,217,868; 6,268,159 and 6,268,390. Such antibodies can be modified or conjugated with various agents such as radioisotopes, toxins, or other cytotoxic agents to enhance their therapeutic effect. Toxins such as ricin and diphtheria toxin, conjugation to liposomes, conjugation to superantigen, and the like are amply described in the literature.

The administration of antibodies for a therapeutic purpose will follow conventional procedures. A liquid formulation is preferred, and can include oils, polymers, vitamins, carbohydrates, amino acids, salts, buffers, albumin, surfactants, or bulking agents. Preferably carbohydrates include sugar or sugar alcohols such as monosaccharides, disaccharides, or polysaccharides, or water soluble glucans. Mannitol is most preferred. The sugars or sugar alcohols can be used individually or in combination. Usually, the sugar or sugar alcohol concentration is between 1.0 w/v % and 7.0 w/v %. Preferably amino acids include L-carnitine, L-arginine, and L-betaine; however, other amino acids can be added. Preferred polymers include polyvinylpyrrolidone with an average molecular weight between 2,000 Da and 3,000 Da, or polyethylene glycol (PEG) with an average molecular weight between 3,000 Da and 5,000 Da. It is also preferred to use a buffer in the composition to minimize pH changes in the solution before lyophilization or after reconstitution. Any physiological buffer can be used, but citrate, phosphate, succinate, and glutamate buffers or mixtures thereof are preferred at a concentration of from about 0.01 M to 0.3 M. Surfactants that can be added to the formulation are descried in European Pat. Nos. 270,799 and 268,110.

Additionally, immunotoxins can be chemically modified by covalent conjugation to a polymer to increase their circulating half-life, for example. Preferred polymers, and methods to attach them to peptides, are shown in U.S. Pat. Nos. 4,766,106; 4,179,337; 4,495,285; and 4,609,546, each of which is incorporated herein by reference, particularly polyoxyethylated polyols and PEG. Water soluble polyoxyethylated polyols, including polyoxyethylated sorbitol, polyoxyethylated glucose, polyoxyethylated glycerol (POG), etc., also are useful in the present invention, with POG preferred.

Another drug delivery system for increasing circulatory half-life utilizes a liposome. Methods of preparing liposome delivery systems are discussed in Gabizon et al., Cancer Res. 42:4734, 1982; Cafiso, Biochem. Biophys. Acta 649:129, 1981; and Szoka, Ann. Rev. Biophys. Eng. 9:467, 1980. Other drug delivery systems are known in the art and are described, for example, in Poznansky et al., “Drug Delivery Systems” (R. L. Juliano, ed., Oxford, N.Y. 1980), pages 253-315; Poznansky, Pharm. Rev. 36:277, 1984.

After the liquid pharmaceutical composition is prepared, it can be lyophilized to prevent degradation and to preserve sterility. Methods for lyophilizing liquid compositions are well known and routine. Just prior to use, the composition can be reconstituted with a sterile diluent such as Ringer's solution, distilled water, or sterile saline, which can include additional ingredients as desired, including additional therapeutic agents specific for or useful for treating a condition. Upon reconstitution, the composition is administered to a subject using any clinical method.

The preferred route of administration is parenterally. In parenteral administration, the compositions of this invention are formulated in a unit dosage injectable form such as a solution, suspension or emulsion, in association with a pharmaceutically acceptable parenteral vehicle. Such vehicles are inherently nontoxic and nontherapeutic. Examples of such vehicles are saline, Ringer's solution, dextrose solution, and Hanks' solution. Nonaqueous vehicles such as fixed oils and ethyl oleate can also be used. A preferred vehicle is 5% dextrose in saline. The vehicle can contain excipients, or additives that enhance isotonicity or chemical stability, including buffers and preservatives.

The dosage and mode of administration will depend on the individual. Generally, the compositions are administered so that the immunotoxins are given at a dose between about 1 μg/kg and 10 mg/kg, generally between about 10 μg/kg and 5 mg/kg, and particularly between about 0.1 mg/kg and 2 mg/kg. The dose can be administered as a bolus dose, or continuous infusion can be used, in which case infusion can proceed at a dose of about 5 Tg/kg/minute to 20 μg/kg/minute, generally about 7 Tg/kg/minute to 15 μg/kg/minute.

An antigen binding fragment of an antibody also can be utilized in the compositions or for practicing the methods of the invention, including, for example, an F(ab), F(ab′)₂, or Fᵥ fragment, as can a variable region of one of the subunit of an Ig, generally the heavy chain subunit, or a chimeric antibody, wherein one of the binding units is specific for one epitope, and the other unit is specific for a different epitope, which can be on the same or a different protein. Each of these forms can serve in a particular situation, depending on the purpose for which the antibody is to be used and the desired outcome.

The antibodies as disclosed herein can be used for a diagnostic or therapeutic purpose, as well as for a general method of detection or purification of the specific active protein. As such, the antibodies can be modified such as by humanization, conjugation with a cytotoxic factor, conjugation with a detectable label such as an enzyme, or fluorescent, chemiluminescent, luminescent, radioactive or paramagnetic moiety. For diagnostic purposes, the antibody can be used for histology, protein determination, cytology, cell classification, and the like. The antibodies can be used in conjunction with other modes of therapy such as viral therapy (see, for example, U.S. Pat. No. 6,136,792), surgery, chemotherapy, hormonal therapy, and the like.

The proteins identified herein and the disclosed antibodies are useful for profiling cells, including cancer cells, which can be at any stage of progression, as to the activity levels of the proteins, particularly serine-threonine hydrolases, in relation to the status of the cancer, at the time of diagnosis, after individual or combined modes of treatment, and the like. The proteins can also be assayed for determining the effect of changes in the environment of the cells on the serine-threonine hydrolases. When evaluating candidate compounds for targets other than prostate cancer, there is an interest to know their effect on prostate cells and the particular proteins that are affected. By use of the antibodies as disclosed herein, the effect of any compound on the activity level of the indicated proteins can be assayed, as can any changes with time after the environment has been changed and either maintained or allowed to revert to an original environment.

The subject probes can also be used in diagnosing the level of PSA in the cells or blood. For diagnosing the level of PSA in the cells, the procedure described above can be used. However, for assaying for PSA in the blood, where the PSA assayed in the active form, a blood sample can be used. The blood sample can be processed by spinning down the cells, filtration, adding citrate, causing clotting, or other such method. The plasma or serum can then be assayed for PSA by adding an appropriate probe under conditions for reaction of the probe with active PSA present in the sample. The amount of probe is sufficient to combine with all of the active PSA in the sample. Since the levels of PSA are known at various stages of prostate cancer, and the amount does not normally exceed 100 μg/ml, usually at least a 2-fold excess of probe is added, and generally not more than about a 10-fold excess is added. The reaction is then allowed to proceed at a temperature in the range of about 25° C. to 40° C. for a sufficient time for at completion of the reaction, generally at least about 15 min and usually not more than about 3 hours. The reaction can be quenched, if desired, by adding a quenching agent such as polycysteine or dithioerythritol. The PSA can then be assayed in a variety of ways, for example, using anti-PSA antibody that is bound to a surface, where the probe comprises a fluorescer; using streptavidin bound to a surface, where the probe comprises biotin, and a labeled anti-PSA antibody then can be added to bind to any PSA present; separation using gel electrophoresis, where the probe comprises a fluorescer; or the like.

The signal from the fluorescer or other detectable label is measured as an indication of the amount of PSA present in the sample. Standards can be employed containing known amounts of PSA and the signal intensity can be plotted against the amount of PSA such that the sample value can be readily determined from the graph, or can be calculated using appropriate algorithms. Total PSA also can be determined using any convenient immunoassay to provide a ratio of active PSA to total PSA. For example, the sample can first be combined with the probe to form a conjugate of probe and PSA, then antibody specific for the probe can be added to sequester the conjugate from the sample, leaving a conjugate-free sample. The antibody conjugate immune complex can then be assayed. Antibody specific for PSA can then be added to the sample and the immune complex of PSA assayed. The amounts of conjugate complex and PSA complex can then be used to determine the active PSA/total PSA ratio.

The present invention also relates to method for determining the status of a prostate epithelial cell, wherein the status is indicative of a normal condition, a hyperplastic condition, or a neoplastic condition. As used herein, the term “status”, when used in reference to prostate epithelial cells, refers to one or more characteristics of the cells. In general, the status is indicative of the condition of the cells, for example, whether the prostate epithelial cells have one or more characteristics of a normal cell or of a cell associated with a proliferative or pathologic condition, particularly a neoplasia, including a benign neoplasm such as benign prostatic hyperplasia and a malignant neoplasm, which can be localized or metastatic. The status of the cells is determined based, for example, on an mRNA profile, protein profile, including total and/or active proteins, spatial distribution profile of the proteins or mRNA, maturity of cells, population of surface membrane proteins, amount and spatial distribution of complexes, amount of ligands present, including bound and/or unbound, lipid population, processing of proteins such as glycosylation, methylation, acylation, phosphorylation, ubiquitination, or farnesylation, and the like.

A method of the invention can be performed, for example, by detecting at least three active serine-threonine hydrolases in prostate epithelial cells, wherein the serine-threonine hydrolases are selected from a fatty acid synthase, a DPP having an apparent molecular mass of about 70 kDa to 95 kDa, a prolyl endopeptidase having an apparent molecular mass of about 71 kDa, a peroxisomal long chain acyl-CoA thioesterase having an apparent molecular mass of about 48 kDa, an epoxide hydrolase having an apparent molecular mass of about 28 kDa, a lysophospholipase-1 having an apparent molecular mass of about 23 kDa, and a protein having an apparent molecular mass of about 60 kDa, wherein the protein is present in normal neoplastic prostate epithelial cells, and is reduced or absent in neoplastic prostate epithelial cells; wherein the presence of at least three of the serine-threonine hydrolases is indicative of a neoplastic condition. The detecting can be performed, for example, by contacting a lysate of the prostate epithelial cell with a probe consisting of a fluorophosphonate group reactive with an active site of a serine-threonine hydrolase joined to a ligand for binding to a receptor or for fluorescence detection by means of an alkylene or oxyalkylene linker, and detecting specific binding of the probe to a serine-threonine hydrolase.

The prostate epithelial cells to be examined, used, or otherwise manipulated according to a method of the invention can be from any organism, particularly a mammalian organism. In general, the prostate epithelial cells are human prostate epithelial cell, such that a method of the invention can, for example, identify a status of the cells characteristic of prostate neoplasia, including benign hyperplasia, and prostate cancer.

The present invention further relates to a method for identifying a compound effective for treating a prostate epithelial neoplasia. Such a screening assay, can be performed, for example, by determining a level of at least serine-threonine hydrolases in a prostate epithelial cell in the presence and absence of the compound, wherein the serine-threonine hydrolases are selected from a fatty acid synthase, a DPP having an apparent molecular mass of from about 70 kDa to 95 kDa, a prolyl endopeptidase having an apparent molecular mass of about 71 kDa, a peroxisomal long chain acyl-CoA thioesterase having an apparent molecular mass of about 48 kDa, an epoxide hydrolase having an apparent molecular mass of about 28 kDa, and lysophospholipase-1 having an apparent molecular mass of about 23 kDa; and detecting a difference in the level of at least three serine-threonine hydrolases in the presence as compared to the absence of the compound. A screening assay of the invention is particularly amenable to a high throughput format, thereby providing a means to screen, for example, a combinatorial library of small organic molecules, peptides, nucleic acid molecules, and the like.

The present invention also provides kits, which can contain any of the compositions disclosed herein or otherwise useful for practicing a method of the invention. As such, a kit of the invention can include, for example, a peptide fragment of a protein disclosed herein as informative of the status of prostate epithelial cells, generally a peptide fragment containing about 10% to 60% of the entire protein, and at least about 12 amino acids, usually at least about 18 amino acids in length; the protein, conveniently in a lyophilized form with stabilizers such as sugars, for example, trehalose; an antibody, which can be in the form of an antiserum, isolated polyclonal antibodies, or monoclonal antibodies, which can further comprise a detectable moiety conjugated thereto. The proteins or fragments thereof can be used as standards for assays for the proteins, can be used conjugated to detectable labels as reagents in assays, where the labeled protein can compete with protein in a sample for an antibody in assays such as fluorescence polarization, and the like.

It will be evident from the present disclosure that biological compositions are provided, including serine-threonine hydrolases, as are antibodies that specifically bind such proteins, including antigen-binding fragments of such antibodies, such reagents be useful for methods of diagnosing and treating prostate cancer. The proteins, antibodies and fragments thereof can be modified by conjugation with a wide variety of other components having differing characteristics for different applications, including labeling with detectable labels, either directly or indirectly, or with entities providing for therapeutic effect. The novel purified proteins can be used to identify prostate cells, evaluate the effect of different therapies, evaluate the effect of drugs having other targets on the expression of these proteins and act as surrogates for evaluating the effect of changes in the environment of prostate cells, including normal, hyperplastic and neoplastic.

The following examples intended to illustrate, but not limit, the present invention.

### EXAMPLE 1

### Serine Hydrolase Signature of Prostate Cancer

This example demonstrates that prostate cancer cell lines display a unique profile, or signature, of active serine hydrolases, and characterizes the molecular identity of these enzymes.

Three well-characterized prostate cancer cell lines were compared to primary cultures of normal prostate epithelial cells, and to three cultures of human fibroblasts. In general, cells were grown in culture, lysed, then the serine hydrolase profiling agents, fp-PEG-Tamra or fp-PEG-Biotin, was added to the lysate. The sample was then separated by SDS-PAGE and labeled serine hydrolases were visualized using a fluorescence gel reader, or by western blot analysis using HRP-avidin. Some labeled serine hydrolases from samples of the prostate cancer cell lines were isolated and identified by mass fingerprinting using MALDI-TOF and MS/MS sequencing.

Methods

Isolation of Cell Lysates

LNCaP, DU-145, and PC-3 prostate cancer cell lines were grown in RPMI 1640 medium supplemented with 10% fetal bovine serum and antibiotics. Normal prostate epithelial cells (PrEC) were grown according to the supplier's instructions (Clonetics).

Confluent monolayers were washed with phosphate buffered saline (PBS) and harvested by scraping cells into PBS. The cells were pelleted at 1,000×g and resuspended in 50 mM Tris, pH 8, and 150 mM NaCl. Following resuspension, the cells were sonicated three times (5 second pulses) at setting 3 using a sonicator ultrasonic processor XL (Heat Systems). The sonicated cell suspensions were lysed with 20 strokes in a Dounce homogenizer.

Soluble and insoluble cell fractions were separated by ultracentrifugation for 1 hr at 64,000 rpm at 4° C. in a Beckman TLC 100.3 rotor. The supernatant containing the soluble fraction was removed, and the remaining insoluble pellet was resuspended in 50 mM Tris, pH 8, and 150 mM NaCl by sonication as described above. The protein concentration of each fraction was measured using the BCA assay (Pierce Chemical Co., Rockford Ill.) according to manufacturer's instructions.

Probing Cell Fractions with Fluorophosphonate Probes

Prior to labeling, each cell fraction was diluted to 1 mg/ml in 50 mM Tris, pH 8, and 150 mM NaCl. The fractions were treated with 50 μl of avidin-agarose (Pierce) to clear endogenously biotinylated proteins. Serine hydrolase activity was profiled using the fluorescent probe, fp-PEG-TAMRA (2 μM) for 1 hr at room temperature (RT). The samples were boiled in Laemmli buffer and resolved on 10% SDS-PAGE gels. The gels were the scanned using a Hitachi FM Bio II fluorescence gel reader and analyzed using the Image Analysis software.

To purify proteins that reacted with the fluorophosphonate probes, the cleared protein suspensions (2 ml) were incubated with 2 TM fp-PEG-biotin for 1 hr at RT. The suspensions containing the probes were passed over a NAP 25 column (Amersham-Pharmacia) to separate proteins from unincorporated fp-PEG-biotin. The pools containing protein were adjusted to 0.5% SDS, by the addition of 10% SDS, and boiled for 10 min. The samples were diluted to a final concentration of 0.2% SDS by the addition of 50 mM Tris, pH 8, and 150 mM NaCl. Avidin agarose (400 Tl) was added, and the suspensions incubated for 1 hr at RT, with rocking. Unlabeled proteins were removed by washing eight times with 50 mM Tris, pH 8, 150 mM NaCl and 1% Triton X-100. Bound proteins, which were labeled with FP-PEG-biotin, were eluted with Laemmli SDS-PAGE loading buffer without glycerol or bromophenol blue. The eluted proteins were concentrated by precipitation by adding 100% acetone at a 3:1 ratio and incubating for 1 hr at −20° C. The precipitated proteins were pelleted by centrifugation at 4° C., resuspended in Laemmli loading buffer, and resolved by SDS-PAGE using 10% pre-cast gels from BioRad.

Identification of Protein Bands by Mass Spectrometry

After SDS-PAGE, the gels were silver stained. Bands of interest were isolated, destained, and subjected to in-gel trypsin digestion (Landry et al., Anal. Biochem. 279, 2000, which is incorporated herein by reference). The tryptic digests of the isolated bands were analyzed by MALDI-TOF using a Voyager DE-RP mass spectrometer (PerSeptive Biosystems; Framingham Mass.). The mass fingerprint was used to query gene and protein databases using the ProFound software (Zhang and Chait, Anal. Chem. 72, 2000; Zhang and Chait, “ProFound-An expert system for protein identification”, In Proceedings of the 46th ASMS Conference on Mass Spectrometry and Allied Topics, Orlando, Fla., 1998.). In several cases, protein identity was obtained, or was confirmed by sequencing individual peptides from the tryptic digest by MS/MS.

Androgen Effects on Cell Proliferation

In order to assess the effects of androgen (DHT) treatment on cell proliferation, LNCaP cells were plated in either 150 mm dishes (Falcon) or in 12 well tissue culture plates (Falcon) in complete medium. After 24 hr, the medium was replaced with RPMI containing no phenol red and supplemented with 10% charcoal stripped fetal bovine serum and the appropriate concentration of DHT. The medium was replaced every 48 hr for six days. At the indicated time points, the cells were removed by treatment with trypsin-EDTA and counted with a hemocytometer.

Profiling Serine Hydrolase Activity in Prostate Cell Lines

Each cell line was grown in 150 mm tissue culture dishes. Prior to collection, the cells were washed with cold PBS, then were harvested by scraping into cold PBS. The collected cells were pelleted by centrifugation. The cell pellets were resuspended in 50 mM Tris-Cl, pH 8.0, 150 mM NaCl. Cell lysis was accomplished by sonication and Dounce homogenization (see above). Following cell lysis, the soluble and insoluble cell fractions were separated by ultracentrifugation for 1 hr at 64,000 rpm at 4° C. The insoluble fractions were further homogenized by sonication. Protein concentrations were determined by BCA assay.

Serine hydrolase activity profiles of the prostate cell lines were measured using the labeled fluorophosphonate probe fp-PEG-Tamra. Briefly, 40 μg of either the soluble or insoluble fractions were treated with 2 μM fp-PEG-Tamra for 1 hr at RT. The labeling reactions were stopped by the addition of Laemmli buffer followed by boiling for 5 min. As a control for non-specific reaction of the probe, a duplicate sample was boiled for 10 min prior to labeling with fp-PEG-Tamra. The labeled samples were resolved by 10% SDS-PAGE and visualized by scanning with a laser at 605 nm.

Inhibition of DPP-Like Activity with Isoleucine Thiozolidide

The sensitivity of enzyme activity to isoleucine-thiozolidide (IT) was tested in the soluble fractions of the LNCaP, DU-145 and PC-3 prostate cancer cells lines. Each lysate was pre-incubated with 100 TM IT for 20 min at RT. Residual DPP-like activity was evaluated by treatment with 2 TM fp-PEG-Tamra, as described above, followed by resolution with 10% SDS-PAGE and comparison to samples not treated with IT. Inhibition of DPP activity was quantified by measuring the fluorescence intensity of the conjugate between fp-PEG-TAMRA and the DPP. The activity of a non-DPP enzyme was used to standardize the specific effects if IT.

Identification of PSA by Immunodepletion and Immunoprecipitation

The identification of prostate specific antigen (PSA) from DHT treated cell lysates was accomplished by antibody subtraction (immunodepletion). Non-specific IgG or anti-PSA monoclonal antibody (Santa Cruz Biotech) were added to LNCaP cell lysates (6 Tg each) and incubated for 4 hr at 4° C. on a rotator. The IgG-protein complexes were precipitated by the addition of 30 Tl protein A/G Plus-Agarose beads (Santa Cruz Biotech) with an additional 1 hr incubation at 4° C. on a rotator. The beads were pelleted by low speed centrifugation and the supernatant was labeled with fp-PEG-Tamra and resolved by SDS-PAGE as described above.

As an alternate strategy, DHT treated LNCaP cell lysates were labeled with fp-PEG-Tamra as described above, then either non-specific mouse IgG or anti-PSA monoclonal antibody (6 μg each) was added to the mixtures. Protein-antibody complexes were formed for 4 hr at 4° C. with rotating. Protein A/G Plus Agarose beads were then added for another hour at 4° C. to precipitate the complexes. The beads were pelleted by low speed centrifugation and washed five times with ice cold 50 mM Tris-Cl, pH 8.0, 150 mM NaCl and 0.2% Tween-20. PSA-fp-PEG-Tamra complexes were eluted by the addition of Laemmli sample buffer and boiling followed by resolution on SDS-PAGE.

Measuring Serine Hydrolase mRNA Levels by Real Time PCR

The serine hydrolase activity levels in the four prostate cell lines, or the change in serine hydrolase activity with DHT treatment, as assessed by fp-PEG-Tamra, was compared to the mRNA level of the cognate enzymes. Total RNA was isolated with Trizol (Life Technologies) from LNCaP, LNCaP treated with DHT, DU-145, PC-3 or PrEC cell lines. Reverse transcription was performed with Superscript II. Real time PCR was performed using the Roche SYBER Green DNA kit and a Roche LightCycler according to manufacturer instructions. Primers (20mers) were used at a final concentration of 5 TM, and 45 cycles were used.

For the activity based profiling of serine hydrolase activity in prostate cell lines with fp-PEG-Tamra, the soluble and insoluble fractions of the total cell lysates of three prostate cancer cell lines (LNCaP, DU-145, and PC-3) and normal prostate epithelial cells (PrEC) were labeled with fp-PEG-Tamra (2 TM) for 1 hr at RT. Following labeling, the samples were boiled in Laemmli sample buffer, and resolved with SDS-PAGE. Specificity of labeling was determined by comparison to preheated controls. Enzymes labeled by the fluorescent probe were identified numerically with an arrow.

Results

The serine hydrolase profiles of the prostate cancer cell lines were remarkably similar to each other, and had some similarity to the profile obtained from the normal prostate epithelial cells. In general, the profiles obtained using the soluble protein fraction were similar, with bands at about 97 kDa, 83 kDa, 81 kDa, 47 kDa, four bands clustering around 30 kDa, and a band of about 26 kDa being common to all cells derived from prostate. In addition, a prominent band of about 217 kDa was observed in all of the prostate cancer cell lines, but was not detected in the normal prostate epithelial cells. In contrast, a major band of about 60 kDa was observed in the normal prostate epithelial cells, but was not detected in any of the prostate cancer cell lines.

In several cases, the molecular identity of these proteins was determined using either peptide mass fingerprinting or MS/MS sequencing (Table 1). One of the proteins unique to the prostate cancer cells, with a mass of about 217 kDa, was determined to be fatty acid synthase, which has been reported to be up-regulated in prostate and breast cancer (Milgraum et al., Clin. Cancer Res. 3:2115-2120, 1997). Inhibition of fatty acid synthase by the natural product, cerulenin, induces apoptosis of tumor cells in culture, and can inhibit tumor growth in nude mice (Pizer et al., The Prostate 47:102-110, 2001; Pizer et al., Cancer Res. 60:213-218, 2000). Fatty acid synthase has multiple activities, all of which coordinate to condense acetyl CoA and malonyl CoA to long chain fatty acids. The final enzymatic step is the hydrolysis of the fatty acid from the acyl carrier protein by a thioesterase, which is a serine hydrolase. The presence of this serine hydrolase within fatty acid synthase is consistent with the ability to label the protein with fp-PEG Tamra, as disclosed herein. Because the amount of fp-PEG-Tamra probe incorporated into the enzyme can be quantified, the number of molecules of fatty acid synthase expressed per cell in an active form also can be determined.

The proteins from the soluble fraction that migrated at about 97 kDa are noteworthy. Bands from the DU-145 and PC3 prostate cancer cells yielded good MALDI mass fingerprints and peptide sequences from MS/MS analysis. By searching the gene databases against the mass fingerprints, and against the results from MS/MS sequencing, genes corresponding to these proteins were identified. Both genes, GenBank Accession Nos. GI:3513303 and GI:3702295, were members of the dipeptidyl peptidase family, though the expression of proteins from these genes does not appear to have been previously reported.

One member of this family, DPP-IV, has been investigated as a target in type II diabetes. As a result of those investigations, several peptidic antagonists of DPP IV have been synthesized (Pederson et al., Diabetes 47:1253-58; 1998; Pauly et al., Metabolism 48(3):385-389, 1999). To further probe the relatedness between these homologs and DPP IV, the ability of one of these antagonists, isoleucine thiazolidide (IT), to block the interaction of these proteins with fp-PEG-Tamra was examined. Inhibition of dipeptidyl peptidase-like activity was examined using isoleucine thiozolidide (IT). The sensitivity of enzyme activity to IT was tested in the soluble fractions of the LNCaP, DU-145, and PC-3 prostate cancer cells lines. Each lysate was pre-incubated with 100 TM IT for 20 min at RT. Residual DPP-like activity was evaluated by the addition of 2 TM fp-PEG-Tamra, followed by resolution with 10% SDS-PAGE and comparison to the non-treated samples. DPP-like activity in the LNCaP, DU-145 and PC-3 cell lines was inhibited 63, 84 and 81%, respectively. No inhibition was seen in non-DPP proteins. Specific labeling was determined by comparison to preheated controls.

Incubation with IT effectively eliminated the binding of the probe to both DPPs. This result demonstrates that IT has broad spectrum antagonist activity for the DPP family. Interestingly, the interaction of fp-PEG-Tamra with the 97 kDa band from the LNCaP prostate cancer cells was also inhibited by IT, indicating that this protein is also a member of the DPP family. These results indicate that the DPP family of serine hydrolases can have a role in the progression of prostate cancer, and can be useful as a target for inhibiting tumor progression using, for example, IT or a similar compound. Table 1 lists the soluble proteins identified from the prostate cell lines and normal prostate cells. Table 2 lists proteins identified in the insoluble fraction of the prostate cell lines and normal prostate cell.

TABLE 1
Serine Hydrolases in Prostate Epithelial Cells (Soluble Cell Fraction)

| Protein Band | LNCaP | DU 145 | PC3 | Normal PrEC |
|---|---|---|---|---|
| (1) 217,000 | Fatty Acid | Fatty Acid | Fatty Acid | Not detected |
| | Synthase | Synthase | Synthase | |
| | (GI: 7433799) | (GI: 7433799) | (GI: 7433799) | |
| (2) 97,000 | unknown | | | |
| (3) 80,000 | Unknown (inhibited | Hypothetical | Hypothetical | |
| | by ILE-TI) | protein | protein | |
| | Presumed DPP | (GI: 3513303) | (GI: 3702295) | |
| | | Presumed DPP | Presumed DPP | |
| (4) 73,000 | N-acylaminoacyl | N-acylaminoacyl | N-acylaminoacyl | |
| | peptide hydrolase | peptide hydrolase | peptide | |
| | (GI: 9951917) | (GI: 9951917) | hydrolase | |
| | | | (GI: 9951917) | |
| (5) 71,000 | Prolyl | Prolyl | Prolyl | |
| | endopeptidase | endopeptidase | endopeptidase | |
| | (GI: 4506043) | (GI: 4506043) | (GI: 4506043) | |
| (6) 60,000 | Not identified | Not identified | Not identified | Human |
| | | | | CarboxylesteraseII |
| (7) 48,000 | Peroxisomal long- | Peroxisomal long- | | |
| | chain acyl-CoA | chain acyl-CoA | | |
| | thioesterase (GI: | thioesterase (GI: | | |
| | 3375614) | 3375614) | | |
| (8) 28,000 | Hypothetical | | | |
| | protein (GI: | | | |
| | 13775216) | | | |
| | Epoxide hydrolase | | | |
| (9) 27,000 | | | | |
| (10) 26,000 | undetectable | undetectable | NOT | undetectable |
| | | | IDENTIFIED | |
| (11) 23,000 | Lysophospholipase-1 | Lysophospholipase-1 | | |
| | (GI: 13654509) | (GI: 13654509) | | |

TABLE 2
Serine Hydrolases in Prostate Epithelial Cells (Insoluble Cell Fraction)

| Protein Band | LNCaP | DU 145 | PC3 | Normal PrEC |
|---|---|---|---|---|
| (1) 217,000 | Fatty Acid | Fatty Acid | Fatty Acid | |
| | Synthase | Synthase | Synthase | |
| | (GI: 7433799) | (GI: 7433799) | (GI: 7433799) | |
| (2) 140,000 | Not detected | Not detected | Not identified | Not detected |
| (3) 80,000 | Unknown | Unknown | Unknown | absent |
| | (inhibited by ILE- | (inhibited by ILE- | (inhibited by | |
| | TI) | TI) | ILE-TI) | |
| | Presumed DPP | Presumed DPP | Presumed DPP | |
| (4) 73,000 | N-acylaminoacyl | N-acylaminoacyl | N- | Not detected |
| | peptide hydrolase | peptide hydrolase | acylaminoacyl | |
| | (GI: 9951917) | (GI: 9951917) | peptide | |
| | | | hydrolase | |
| | | | (GI: 9951917) | |
| (5) 57,000 | absent | absent | absent | unknown |
| (6) 56,000 | absent | absent | absent | unknown |
| (7) 55,000 | absent | absent | absent | unknown |
| (8) 48,000 | Peroxisomal long- | Hypothetical | | |
| | chain acyl-CoA | Protein | | |
| | thioesterase (GI: | GI: 7243107 | | |
| | 3375614) | | | |
| (9) 45,000 | | | | |
| (10) 30,000 | | | | |
| (11) 28,000 | Hypothetical | | | |
| | proteins (GI: | | | |
| | 3775216 and | | | |
| | GI: 8923001) | | | |
| | Epoxide hydrolase | | | |
| (12) 26,000 | | | | |
| (13) 23,000 | Lysophospholipase-1 | | | |
| | (GI: 13654509) | | | |

Defining Aggregate Serine Hydrolase Activity Profile of Prostate Cancer Cell Lines

As the first step toward understanding the role of serine hydrolases in the progression of prostate cancer, the aggregate serine hydrolase profile of the LNCaP, DU-145 and PC-3 prostate cancer cell lines was profiled. The PrEC normal prostate cell line was used as representative of normal prostate cells. The soluble and insoluble, or membrane, fractions were separated and profiled independently with the fluorescent probe fp-PEG-Tamra. Approximately 10 to 13 bands were present in the soluble fraction of each cell lysate, indicating that there was the same number of active serine hydrolases in the different cell lines. In general, the aggregate profile of the three prostate cancer cell lines appeared the same. However, there were differences in the activities of the individual hydrolases between the three cell lines. The PrEC cell line exhibited a number of differences from the three cancer cell lines, the most obvious difference being the presence of a band of about 52 kDa, which was absent in the three cancer cell lines. In addition, there were several enzymes that were reduced or absent in the PrEC profile as compared to the three cancer cell lines (for example, the 200 kDa and 90 kDa bands).

The profiles of the insoluble, or membrane, fractions of the four cell lines showed more divergence than was observed in the soluble profiles. The number of bands, which represent active enzymes, in the gels of the soluble proteins ranged from 7 to 17. However, the magnitude of difference between the PrEC cell line and the three cancer cell lines was greater in the insoluble fraction than in the soluble fraction. While bands of the same molecular weight were seen in the both the soluble and insoluble fractions, there were a number of unique enzyme activities in the insoluble fractions when compared to the soluble fractions. This was especially true in the 30 kDa to 40 kDa range, where most of the differences were observed. In general, though, most of the enzyme activity that was present in the three cancer cell lines was reduced or absent in the normal PrEC cells. Likewise, most of the bands that were present in the PrEC cells appeared to be reduced or absent in the cancer cell lines. These results demonstrate that a fingerprint of serine hydrolase activity can distinguish phenotypic differences between normal prostate cells and prostate cancer cell lines.

Identification of Serine Hydrolases in Prostate Cell Lines

The comparison of the serine hydrolase fingerprint, or aggregate activity profile, of the three prostate cancer cell lines and the normal PrEC cell line illustrated some dramatic differences between the cell lines. In order to understand how the difference in activity profiles might be translated to biological function, MALDI-TOF spectroscopy and MS/MS sequencing were used to identify the serine hydrolases described above. To purify the serine hydrolases for identification, cell lysates were labeled with fp-PEG-biotin, then the biotin labeled enzymes were enriched by avidin-biotin affinity chromatography.

The range of enzymes identified from the soluble and insoluble fractions of the prostate cell lines was as varied as might be expected of the serine hydrolase family. In the soluble fraction, a number of the identified enzymes were common in all three cancer cell lines, including fatty acid synthase, N-acyl peptide hydrolase, proly endopeptidase, long chain CoA thioesterase, and lysophospholipase. Although these enzymes were common to all three cancer cell lines, the relative activity of each enzyme differed among the cell lines. Interestingly, the bands migrating in the range of about 70 kDa to 95 kDa in the soluble fraction appeared to be homologs of dipeptididyl peptidase (DPP). The homologs were unique and different in each of the three cell lines, despite their apparent similarity in molecular weight (see below).

The profile of the PrEC cells was distinct from that of either of the three cancer cell lines. The most obvious difference was the appearance of the band at about 52 kDa. The other telling differences were the absence of fatty acid synthase and N-acyl peptide hydrolase. The absent, or reduced, activity of these enzymes indicates that their enzymatic functions are not as critical to growth and survival of the normal prostate cells as they are in the cancer cell lines.

The activity profile illustrated dramatic differences between the insoluble fractions of the four cell lines tested. For the most part, all of the enzymes identified by MALDI or MS/MS in the insoluble fraction were identical to those identified in the soluble fractions. This result was likely due to the fact that the enzymes either localize to regions that do not partition well between fractions, or due to incomplete separation of the fractions. Moreover, there were a number of enzymes between about 30 kDa and 40 kDa that could not be identified due to their abundance or to purification complications.

Inhibition of DPP Activity by Isoleucine Thiozolidide

In each of the three prostate cancer cell lines, a novel protein with homology to DPP was identified using MS/MS. The similarity to DPP was based on fold and function assignment of the corresponding cDNA and its cognate protein (Zhang et al., Prot. Sci. 8:1104-1115, 1999). In order to obtain a functional assessment of DPP activity by these proteins, the soluble fraction of cell lysates from the three cell lines was treated with isoleucine thiozolidide (IT), a known inhibitor of DPP activity, to block the complex with fp-PEG-Tamra. The DPP-like activity in the LNCaP, DU-145 and PC-3 cell lines was inhibited 63, 84 and 81%, respectively, following pre-treatment with IT. The inhibition by IT was specific, as no other serine hydrolase activity was reduced.

Effects of Androgen on Serine Hydrolase Activity

It is well established that androgen (DHT) specifically, promotes proliferation of LNCaP cells in vitro. While the direct cause for this is not completely understood, it is clear that changes in gene and protein levels are associated with proliferation. As such, it was hypothesized that androgen treatment of LNCaP cells would change the aggregate activity profile. To test this hypothesis, cells were treated with two DHT concentrations over a course of six days, and the effect of DHT on cell proliferation and aggregate serine hydrolase activity was examined. LNCaP cell proliferation was measured in the presence of either 0.1 nM or 100 nM DHT. Cells were plated in 24 well plates in RPMI 1640 with no phenol red and supplemented with charcoal stripped FBS and allowed to adhere overnight. The following day media was replaced, with or without DHT, and cells were grown for six days with media change every second day. Cells were counted using a hemocytometer. On day six, the aggregate serine hydrolase activity profiles were also measured. Hydrolase activity was resolved by 10% SDS-PAGE. Specific labeling was assessed by comparison to a preheated control.

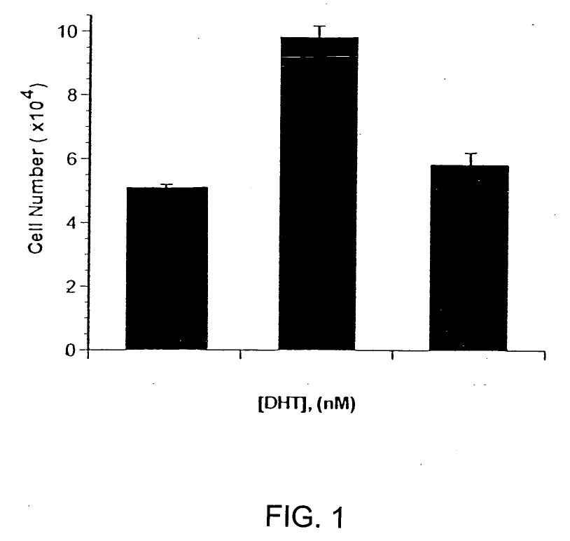

As expected, treatment of LNCaP cells with a low concentration of DHT (0.1 nM) promoted cell proliferation, while treatment with a high concentration of DHT (100 nM) inhibited proliferation (FIG. 1). Concomitant with these effects, a change in the serine hydrolase profile was observed at each DHT concentration.

When LNCaP cell were treated with 0.1 nM DHT over a course of six days, the activity levels of two serine hydrolases changed. The activity of fatty acid synthase (FAS) increased three-fold to five-fold with this concentration of DHT. This result was not surprising because FAS activity is associated with cell proliferation. Interestingly, the level of FAS activity increased more than the change in mRNA level would indicate. An opposite effect on activity was seen with prolyl endopeptidase (PEP). PEP activity decreased after treatment with 0.1 nM DHT for six days. The change in mRNA level for PEP was in accordance with the change in activity levels. The biggest discrepancy between activity level and mRNA level at this DHT concentration was found with PSA. Although the PSA mRNA level increased over eleven-fold, there was no noticeable change in activity levels. This result indicates that the amount of active PSA was still below detectable levels in these samples.

The scope and magnitude of changes in the serine hydrolase activity profile of LNCaP cells was much greater when the cells were treated with 100 nM DHT. At this concentration, the activity profiles of at least four serine hydrolases were affected. Similar to what was observed with the 0.1 nM DHT treated cells, FAS activity increased, although above the levels observed in the 0.1 nM treated samples. This result was surprising because cell proliferation was inhibited at this DHT concentration. However, the inhibited proliferation can be explained by paracrine effects of other molecules induced by this concentration of DHT that do not effect FAS expression or activity. As expected the level of PSA activity also increased with this DHT treatment, and there was a large increase in PSA mRNA levels.

The change in the LNCaP serine hydrolase activity profile following treatment with 100 nM DHT was also characterized by a dramatic drop in both PEP and N-acyl peptide hydrolase (NAPH) activity. These data correlated well with the inhibited cell proliferation observed with this concentration of DHT, as these hydrolases are associated with processing of growth factors or protein turnover. The activity profiles of the two enzymes were not consistent with their mRNA level; in both cases the cognate mRNA level was increased following treatment with 100 nM DHT. In the case of NAPH the increase was slightly greater than two-fold. These results indicate that factors aside from mRNA levels that regulate the catalytic activity of these proteins.

Identification of Prostate Specific Antigen (PSA) by Immunodepletion

The serum level of PSA is the most common biomarker for the diagnosis of prostate cancer. In addition, it is known that PSA levels increase in LNCaP cell following DHT treatment. Because of this, and the fact that a new band of serine hydrolase activity of about 35 kDa appeared in the LNCaP activity profile after treatment with 100 nM DHT, the 35 kDa band was examined to determine whether it was PSA. LNCaP cells were treated with 100 nM DHT for six days, then lysed. The lysate was incubated with either an anti-PSA monoclonal antibody or non-specific mouse IgG. The IgG-protein complexes were precipitated with protein Plus-A/G SEPHAROSE gel, and the remaining serine hydrolase activity was profiled.

Prostate specific antigen (PSA) was identified by immunodepletion and immunoprecipitation. LNCaP cell were treated with or without DHT (100 nM) for six days. To identify PSA by immunodepletion, lysates were treated with either anti-PSA mAB or non-specific IgG. The IgG-protein complexes were removed with protein PLUS A/G agarose beads and low speed centrifugation. Remaining hydrolase activity was profiled with fp-PEG-Tamra. PSA was also identified by immunoprecipitation of PSA-fp-PEG-Tamra complexes. The soluble fraction of lysates from LNCaP cell treated with or without DHT (100 nM) were labeled with fp-PEG-Tamra. Immunoprecipitation was performed by the addition of either non-specific mouse IgG or anti-PSA mAb. The IgG-protein complexes were removed by the addition of protein Plus A/G agarose beads and low speed centrifugation. Following washing, the precipitated activity was eluted by the addition of Laemmli buffer and boiling and resolved by SDS-PAGE.

As expected, the band at 35 kDa was no longer present in the activity profile after treatment with anti-PSA antibody. On the other hand, non-specific mouse IgG had no effect on the profile, indicating that the protein is indeed PSA.

As a further confirmation of the identity of PSA, cell lysate from LNCaP cells treated with 100 nM DHT was labeled with fp-PEG-Tamra, then the labeled mixture was treated with either non-specific mouse IgG or anti-PSA monoclonal antibody. The IgG-protein complexes were precipitated with protein plus-A/G-SEPHAROSE gel and the beads were boiled in Laemmli buffer. The resulting samples that were eluted from the beads were resolved by SDS-PAGE. Only the sample that had been treated with anti-PSA antibody showed a band indicating activity at 35 kDa, thus confirming that the 35 kDa protein was PSA. Moreover, this result demonstrates a method of using fp-PEG-Tamra-PSA complexes as a non-ELISA-based method of identifying PSA activity in biological samples.

### EXAMPLE 2

### Fluorescent Probes

This example provides methods for preparing fluorescent probes useful for profiling a proteome.

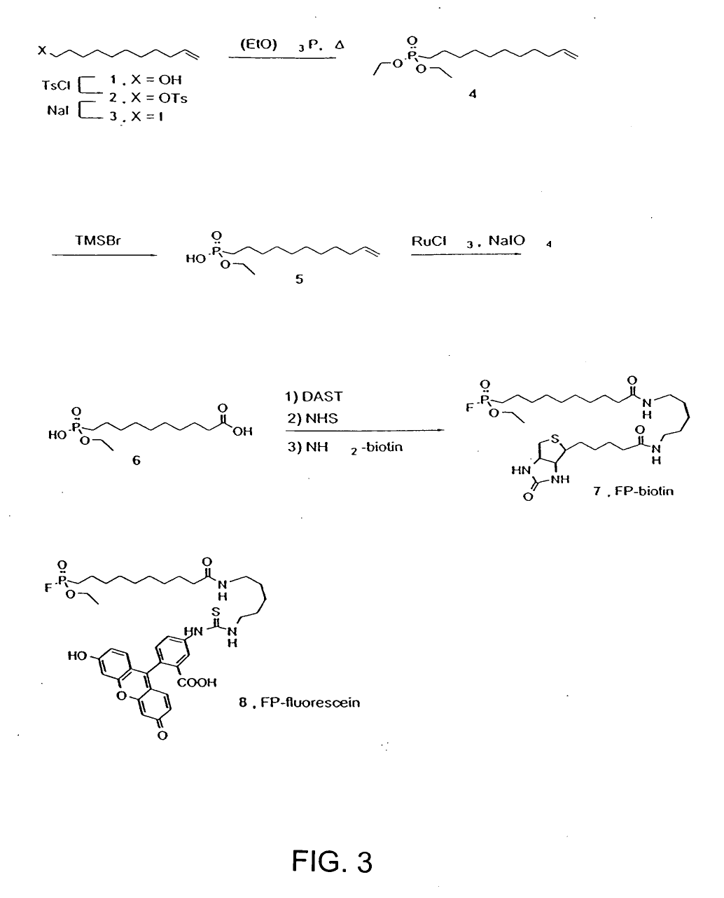

Compound 1a is the starting material tetraethyleneoxy (3,6,9-oxa-1,11-diolundecane) and compound 1b is the starting material decylene-1,10-diol as depicted in the flow chart in FIG. 3. Preparation of triethyleneoxy-linked fluorophosphonate and N-fluorescer-formamidoalkylenecarbamoyl (fluorescer is BodipyFL or tetramethylrhodamine and the alkylene is 2 or 5 carbon atoms respectively), or N-fluorescein thioureidopentanylcarbamoyl, where the fluorescer in this example is fluorescein. The other fluorescer compounds are made in substantially the same way, using the different fluoresceralkylamino derivatives as shown in the flow chart.

Compound 2. A solution of 1 (3.9 g, 20.0 mmol, 3.0 equiv) in DMF (8.0 ml) was treated with TBDMSCl (1.0 g, 6.64 mmol, 1.0 equiv) and imidazole (0.9 g, 13.3 mmol, 2.0 equiv) and the reaction mixture was stirred for 12 hr at RT. The reaction mixture was then quenched with saturated aqueous NaHCO₃ and partitioned between ethyl acetate (200 ml) and water (200 ml). The organic layer was washed with dried (Na₂SO₄) and concentrated under reduced pressure. Chromatography (SiO₂, 5×15 cm, 50-100% ethyl acetate-hexanes) afforded 2 (1.1 g, 2.0 g theoretical, 55%) as a colorless oil: 1H NMR (CDCl₃, 400 MHz) δ 3.8-3.5 (m, 16H, CH₂OR), 0.88 (s, 9H, CH₃C), 0.0 (s, 6H, CH₃Si).

Compound 3. A solution of 2 (0.61 g, 2.0 mmol, 1.0 equiv) in benzene (15 ml, 0.13 M) was treated sequentially with PPh3 (2.6 g, 10.0 mmol, 5 equiv), 12 (2.3 g, 9.0 mmol, 4.5 equiv), and imidazole (0.7 g, 10.3 mmol, 5.2 equiv) and the reaction mixture was stirred at room temperature for 30 min, producing a yellow-orange heterogeneous solution. The soluble portion of the reaction mixture was removed and the insoluble portion washed several times with ethyl acetate. The combined reaction and washes were then partitioned between ethyl acetate (200 ml) and saturated aqueous Na₂S₂O₃ (200 ml). The organic layer was washed sequentially with H₂O (100 ml) and saturated aqueous NaCl (100 ml), dried (Na₂SO₄), and concentrated under reduced pressure. Chromatography (SiO2, 5×15 cm, 5-25% ethyl acetate-hexanes) afforded 3 (0.54 g, 0.82 g theoretical, 66%) as a colorless oil: 1H NMR (CDCl₃, 400 MHz) δ 3.85-3.60 (m, 12H, CH₂OR), 3.54 (t, J=5.6, 2H, CH₂OTBDMS), 3.23 (t, J=7.0 Hz, 2H, CH₂I), 0.88 (s, 9H, CH₃C), 0.0 (s, 6H, CH₃Si).

Compound 4. Triethylphosphite (1.2 mL, 7.0 mmol, 5.4 equiv) was added to 3 (0.53 g, 1.29 mmol, 1.0 equiv) and the mixture was stirred at 150° C. for 1 hr. The reaction mixture was cooled to RT and directly submitted to flash chromatography (SiO2, 5×15 cm, 100% ethyl acetate) to afford 4 (0.43 g, 0.54 g theoretical, 80%) as a colorless oil: 1H NMR (CDCl₃, 400 MHz) δ 4.20-4.05 (m, 4H, CH₃CH₂OP), 3.80-3.55 (m, 14H, CH₂OR), 2.15 (m, 2H, CH₂P), 1.31 (t, J=6.0 Hz, 6H, CH₃CH₂OP), 0.88 (s, 9H, CH₃C), 0.0 (s, 6H, CH₃Si).

Compound 5. A solution of compound 4 (0.21 g, 0.5 mmol, 1.0 equiv) in CH2Cl2 (2.8 ml, 0.18 M) was treated with HF-pyridine (0.084 mL, ˜0.84 mmol, approximately 1.7 equiv). The reaction was stirred at 25° C. for 30 min, then partitioned between ethyl acetate (100 ml) and water (100 ml). The organic layer was dried (Na₂SO₄) and concentrated under reduced pressure. Chromatography (SiO2, 2×8 cm, 3-10% CH3OH—CH₂Cl₂) afforded 5 (0.050 g, 0.28 g theoretical, 32.5%) as a clear oil: 1H NMR (CDCl₃, 400 MHz) δ 4.20-4.05 (m, 4H, CH3CH₂OR), 3.80-3.55 (m, 14H, CH₂OR), 2.15 (m, 2H, CH2P), 1.31 (t, J=6.0 Hz, 6H, CH3CH2OP); MALDI-FTMS m/z 337.1377 (C12H27O7P+Na+requires 337.1387).

Compound 6. A solution of 5 (0.030 g, 0.096 mmol, 1.0 equiv) in DMF (0.28 ml, 0.34 M) was treated sequentially with N,N-disuccinimidyl carbonate (0.058 g, 0.22 mmol, 2.2 equiv) and triethylamine (0.035 μL, 0.25 mmol., 2.5 equiv). The reaction mixture was stirred at RT for 12 hr, then partitioned between CH₂Cl_{2 (}100 ml) and H2O (100 ml). The organic layer was washed with saturated aqueous NaCl (100 ml), dried (Na₂SO₄), and concentrated under reduced pressure. Chromatography (SiO2, 2×8 cm, 1-10% CH3OH—CH₂Cl₂) afforded 50.035 g, 0.043 g theoretical, 81%) as a clear oil: 1H NMR (CDCl₃, 400 MHz) δ 4.45 (m, 2H, CH2OC(O)OR), 4.20-4.05 (m, 4H, CH3CH2OP), 3.80-3.55 (m, 12H, CH2OR), 2.84 (s, 4H, CH2C(O)N), 2.15 (m, 2H, CH2P), 1.31 (t, J=6.0 Hz, 6H, CH3CH2OP). MALDI-FTMS m/z 478.1456 (C17H30NO11P+Na+ requires 478.1449).

Compound 7. A solution of 6 (0.020 g, 0.044 mmol, 1.0 equiv) in CH2Cl2 (0.14 ml, 0.40 M) was cooled to 0° C. and treated with oxalyl chloride (0.082 mL, 2 M in CH2Cl2, 0.164 mM 3.7 equiv). The reaction mixture was allowed to warm to RT and stirred for 18 hr. The reaction mixture was then concentrated under a stream of gaseous nitrogen and the remaining residue treated with H2O (0.1 ml) for 5 min. The H2O was evaporated under a stream of gaseous nitrogen and the remaining residue dried by vacuum to provide 7 (0.015 mg, 0.019 mg theoretical, 80%) as a clear oil/film: 1H NMR (CDCl₃, 400 MHz) δ 4.45 (m, 2H, CH2OC(O)OR), 4.10 (m, 2H, CH3CH2OP), 3.80-3.55 (m, 12H, CH2OR), 2.84 (s, 4H, CH2C(O)N), 2.15 (m, 2H, CH2P), 1.31 (t, J=6.0 Hz, 3H, CH3CH2OP).

Compound 8. A solution of 7 (0.007 g, 0.016 mmol, 1.0 equiv) in CH₂Cl₂ (0.22 ml, 0.075 M) at −78° C. was treated with (diethylamino)sulfur trifluoride (DAST, 0.007 ml, 0.048 mmol, 3.0 equiv) and the reaction mixture was stirred for 10 min. The reaction mixture was then partitioned between ethyl acetate (100 ml) and H2O (100 ml) and the organic layer was washed with saturated aqueous NaCl (100 ml;), dried (Na2SO4), and concentrated under reduced pressure. Chromatography (SiO2, Pasteur pipette, 100% ethyl acetate) afforded 8 (0.003 g, 0.007 g theoretical, 42%) as a clear oil: 1H NMR (CDCl3, 400 MHz) δ 4.45 (m, 2H, CH2OC(O)OR), 4.27 (m, 2H, CH3CH2OP), 3.80-3.55 (m, 12H, CH2OR), 2.84 (s, 4H, CH2C(O)N), 2.32-2.26 (m, 2H, CH2P), 1.31 (t, J=6.0 Hz, 3H, CH3CH2OP).

Compound 9. A solution of tetramethylrhodamine cadaverine (Molecular Probes; Eugene Oreg.) (0.005 g, 0.010 mmol, 1.0 equiv) in DMF (0.5 ml, 0.020 M) was added to compound 8 (neat, 0.007 g, 0.016 mmol, 1.7 equiv) and the reaction mixture was stirred for 30 min at RT. The solvent was removed under vacuum and the products were resuspended in a 0.35 mL of a water-acetonitrile mixture (1:1 v./v.) containing 0.1% (v./v.) trifluoroacetic acid. An aliquot of this solution (0.30 ml) was injected on a preparative reverse phase HPLC column (Haisil 100 C8, Higgins Analytical, 20 mm×150 mm), separated using a 0-100% acetonitrile gradient in 30 min at 10 ml per min. The retention time under these conditions was 19.95 min. The solvent was removed under vacuum using a rotary evaporator, and afforded 9 (0.0035 g, 0.0042 mmol, 42%) as a darkly colored oil.

FP-alkyleneamino-fluorescer was prepared as described by Liu et al. (Proc. Natl. Acad. Sci., USA 96(26):14694, 1999; see, also, PCT/US00/34187, each of which is incorporated herein by reference. 1-Iodo-10-undecene (3). A solution of 2 (3.4 g, 10.5 mmol, 1.0 equiv) in acetone (21 ml, 0.5 M) was treated with NaI (3.2 g, 21 mmol, 2.0 equiv) and the reaction mixture was stirred at reflux for 2 hr, producing a yellow-orange solution. The reaction mixture was then partitioned between ethyl acetate (200 ml) and water (200 ml). The organic layer was washed sequentially with saturated aqueous Na2S2O3 (100 ml) and saturated aqueous NaCl (100 ml), dried (Na2SO4), and concentrated under reduced pressure. Chromatography (SiO2, 5×15 cm, 1-2% ethyl acetate-hexanes) afforded 3 (2.3 g, 2.9 g theoretical, 78%) as a colorless oil: 1H NMR (CDCl3, 250 MHz) δ 5.95-5.75 (m, 1H, RCH═CH2), 5.03-4.90 (m, 2H, RCH═CH2), 3.16 (t, J=7.0 Hz, 2H, CH2I), 2.02 (m, 2H, CH2CH═CH2), 1.80 (p, J=6.9 Hz, 2H, CH2CH2I), 1.50-1.20 (m, 12H).

1-{Bis(ethoxy)phosphinyl}-10-undecene (4). Triethylphosphite (12.2 ml, 71 mmol, 10 equiv) was added to 3 (2.0 g, 7.1 mmol, 1.0 equiv) and the mixture was stirred at reflux for 15 hr. The excess triethylphosphite was removed by distillation and the remaining residue submitted to flash chromatography (SiO2, 5×15 cm, 25-50% ethyl acetate-hexanes gradient elution) to afford 4 (1.30 g, 2.1 g theoretical, 62%) as a colorless oil: 1H NMR (CDCl3, 250 MHz) δ 5.95-5.75 (m, 1H, RCH═CH2), 5.03-4.90 (m, 2H, RCH═CH2), 4.05 (m, 4H, CH3CH2OP), 2.02 (m, 2H, CH2CH═CH2), 1.80-1.20 (m, 20H); MALDI-FTMS (DHB) m/z 291.2088 (C15H31O3P+H+ requires 291.2089).

1-(Ethoxyhydroxyphosphinyl)-10-undecene (5). A solution of compound 4 (0.31 g, 1.07 mmol, 1.0 equiv) in CH2Cl2 (4.0 mL, 0.3 M) was treated dropwise with trimethylsilyl bromide (TMSBr, 0.17 ml, 1.28 mmol, 1.2 equiv). The reaction was stirred at 25° C. for 1 hr, quenched with 5 ml of 5% (w/v) KHSO4, and stirred vigorously for 5 min. The reaction mixture was then partitioned between ethyl acetate (100 ml) and water (100 ml), and the organic layer was washed with saturated aqueous NaCl (200 ml), dried (Na2SO4), and concentrated under reduced pressure. Chromatography (SiO2, 2×8 cm, 12-20% CH3OH—CHCl3 with 1% aqueous NH4OH) afforded 5 (0.10 g, 0.28 g theoretical, 36.2. %; most of the remaining mass was recovered as starting material) as a clear oil: 1H NMR (CDCl3, 250 MHz) δ 5.95-5.75 (m, 1H, RCH═CH2), 5.03-4.90 (m, 2H, RCH═CH2), 4.05 (m, 2H, CH3CH2OP), 2.02 (m, 2H, CH2CH═CH2), 1.80-1.20 (m, 20H). MALDI-FTMS (DHB) m/z 285.1589 (C13H27O3P+Na+ requires 285.1596).

10-(Ethoxyhydroxyphosphinyl)-decanoic acid (6). Compound 5 (0.10 g, 0.38 mmol, 1.0 equiv) in a biphasic solution composed of CCl4/CH3CN/H2O (1.0 ml/1.0 ml/1.5 ml; total volume of 3.5 ml, 0.11 M) was treated sequentially with sodium periodate (0.31 g, 1.56 mmol, 4.1 equiv) and ruthenium trichloride hydrate (0.002 g, 0.009 mmol, 0.022 equiv). The reaction mixture was stirred at 25° C. for 2 hr, then partitioned between CH2Cl2 (50 ml) and 1 N aqueous HCl (50 ml). The organic layer was washed with saturated aqueous NaCl (25 ml), dried (Na2SO4), and concentrated under reduced pressure. The resulting residue was resuspended in 40 ml of diethyl ether, filtered through a Celite pad, and concentrated under reduced pressure to afford 6 (0.09 g, 0.11 g theoretical, 83%) as a colorless semisolid:

1H NMR (CDCl3, 250 MHz) δ 4.05 (m, 2H, CH3CH2OP), 2.32 (t, J=7.5 Hz, 2H, CH2COOH), 1.80-1.20 (m, 16H); FABHRMS (NBA-NaI) m/z 303.1340 (C12H25O5P+Na+ requires 303.1337).

FP-fluorescer, or 10-(fluoroethoxyphosphinyl)-N-(fluoresceramidopentyl)-decanamide (7). A solution of 6 (0.007 g, 0.025 mmol, 4.0 equiv) in CH2Cl2 (0.4 ml, 0.06 M) at −78° C. was treated dropwise with (diethylamino)sulfur trifluoride (DAST, 0.021 mL, 0.10 mmol, 4.0 equiv), brought to 25° C., and stirred for 5 min. The reaction mixture was treated with one-half reaction volume of dimethyl formamide containing N-hydroxysuccinimide (0.05 g, 0.25 mmol, 10 equiv) and stirred for an additional 10 min at 25° C. The reaction mixture was then partitioned between ethyl acetate (50 ml) and water (50 ml), and the organic layer was washed with saturated aqueous NaCl (200 ml), dried (Na2SO4), and concentrated under reduced pressure to afford 110-(fluoroethoxyphosphinyl)-N-(hydroxysuccinyl)-decanamide (as judged by crude 1H NMR;). Without further purification, this compound was treated with 5-(fluoresceramido)-pentylamine (Pierce, 0.0021 g, 0.062 mmol, 1.0 equiv) in MeOH (0.02 ml) and stirred for 10 min. The solvent was evaporated under a stream of gaseous nitrogen and the remaining residue was washed sequentially with diethyl ether and ethyl acetate, solubilized in a minimal volume of chloroform, transferred to a clean glass vial, and the solvent evaporated. This process was repeated once more to rid the desired product of excess reagents and byproducts, affording the desired product in substantially pure form.

Although the invention has been described with reference to the above example, it will be understood that modifications and variations are encompassed within the spirit and scope of the invention. Accordingly, the invention is limited only by the following claims.

1-14. (canceled)

15. A method for identifying a compound effective for treating a prostate epithelial neoplasia, the method comprising
a) determining a level of activity of at least three serine-threonine hydrolases in a prostate epithelial cell in the presence and absence of the compound,
wherein the serine-threonine hydrolases are selected from:
a fatty acid synthase,
a dipeptidyl peptidase having an apparent molecular mass of from about 70 kDa to 95 kDa,
a prolyl endopeptidase having an apparent molecular mass of about 71 kDa,
a peroxisomal long chain acyl-CoA thioesterase having an apparent molecular mass of about 48 kDa,
an epoxide hydrolase having an apparent molecular mass of about 28 kDa, and
lysophospholipase-1 having an apparent molecular mass of about 23 kDa; and
b) detecting a difference in the level of activity of at least three serine-threonine hydrolases in the presence as compared to the absence of the compound, thereby identifying a compound effective in treating the prostate epithelial neoplasia.

16. The method of claim 15, wherein at least one of said three serine-threonine hydrolases is a dipeptidyl peptidase, and wherein the dipeptidyl peptide is not dipeptidyl peptidase IV.

17. The method of claim 15, wherein the prostate epithelial cell is a human prostate epithelial cell.

18-30. (canceled)


SEQUENCE LISTING (19 sequences)

SEQ ID NO: 1 - protein, 2509 aa, Homo sapiens [sequence omitted]

SEQ ID NO: 2 - DNA, 8460 bp, Homo sapiens
  misc_feature (8148)..(8148): n is any nucleotide
cggccgtcgacacggcagcggccccggcctccctctccgccgcgcttcagcctcccgctccgccgcgctccagcctcgctctccgccgcccgcaccgccgcccgcgccctcaccagagcagccatggaggaggtggtgattgccggcatgtccgggaagctgccagagtcggagaacttgcaggagttctgggacaacctcatcggcggtgtggacatggtcacggacgatgaccgtcgctggaaggcggggctctacggcctgccccggcggtccggcaagctgaaggacctgtctaggtttgatgcctccttcttcggagtccaccccaagcaggcacacacgatggaccctcagctgcggctgctgctggaagtcacctatgaagccatcgtggacggaggcatcaacccagattcactccgaggaacacacactggcgtctgggtgggcgtgagcggctctgagacctcggaggccctgagccgagaccccgagacactcgtgggctacagcatggtgggctgccagcgagcgatgatggccaaccggctctccttcttcttcgacttcagagggcccagcatcgcactggacacagcctgctcctccagcctgatggccctgcagaacgcctaccaggccatccacagcgggcagtgccctgccgccatcgtggggggcatcaatgtcctgctgaagcccaacacctccgtgcagttcttgaggctggggatgctcagccccgagggcacctgcaaggccttcgacacagcggggaatgggtactgccgctcggagggtgtggtggccgtcctgctgaccaagaagtccctggcccggcgggtgtacgccaccatcctgaacgccggcaccaatacagatggcttcaaggagcaaggcgtgaccttcccctcaggggatatccaggagcagctcatccgctcgttgtaccagtcggccggagtggcccctgagtcatttgaatacatcgaagcccacggcacaggcaccaaggtgggcgacccccaggagctgaatggcatcacccgagccctgtgcgccacccgccaggagccgctgctcatcggctccaccaagtccaacatggggcacccggagccagcctcggggctggcagccctggccaaggtgctgctgtccctggagcacgggctctgggcccccaacctgcacttccatagccccaaccctgagatcccagcgctgttggatgggcggctgcaggtggtggaccagcccctgcccgtccgtggcggcaacgtgggcatcaactcctttggcttcgggggctccaacgtgcacatcatcctgaggcccaacacgcagccgccccccgcacccgccccacatgccaccctgccccgtctgctgcgggccagcggacgcacccctgaggccgtgcagaagctgctggagcagggcctccggcacagccaggacctggctttcctgagcatgctgaacgacatcgcgctgtccccgaccaccgccatgcccttccgtggctacgctgtgctgggtggtgagcgcggtggcccagaggtgcagcaggtgcccgctggcgagcgcccgctctggttcatctgctctgggatgggcacacagtggcgcgggatggggctgagcctcatgcgcctggaccgcttccgagattccatcctacgctccgatgaggctgtgaaccgattcggcctgaaggtgtcacagctgctgctgagcacagacgagagcacctttgatgacatcgtccattcgtttgtgagcctgactgccatccagataggcctcatagacctgctgagctgcatggggctgaggccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccggcggtggtgcccgcctgccacaactccaaggacacagtcaccatctcgggacctcaggccccggtgtttgagttcgtggagcagctgaggaaggagggtgtgtttgccaaggaggtgcggaccggcggtatggccttccactcctacttcatggaggccatcgcacccccactgctgcaggagctcaagaaggtgatccgggagccgaagccacgttcagcccgctggctcagcacctctatccccgaggcccagtggcacagcagcctggcacgcacgtcctccgccgagtacaatgtcaacaacctggtgagccctgtgctgttccaggaggccctgtggcacgtgcctgagcacgcggtggtgctggagatcgcgccccacgccctgctgcaggctgtcctgaagcgtggcctgaagccgagctgcaccatcatccccctgatgaagaaggatcacagggacaacctggagttcttcctggccggcatccggaggctgcacctctcaggcatcgacgccaaccccaatgccttgttcccacctgtggagttcccagctccccgaggaactcccctcatctccccactcatcaagtgggaccacagcctggcctgggacgtgccggccgccgaggacttccccaacggttcaggttccccctcagccgccatctacaacatcgacaccagctccgagtctcctgaccactacctggtggaccacaccctcgacggtcgcgtcctcttccccgccactggctacctgagcatagtgtggaagacgctggcccgacccctgggcctgggcgtcgagcagctgcctgtggtgtttgaggatgtggtgctgcaccaggccaccatcctgcccaagactgggacagtgtccctggaggtacggctcctggaggcctcccgtgccttcgaggtgtcagagaacggcaacctggtagtgagtgggaaggtgtaccagtgggatgaccctgaccccaggctcttcgaccacccggaaagccccacccccaaccccacggagcccctcttcctggcccaggctgaagtttacaaggagctgcgtctgcgtggctacgactacggccctcatttccagggcatcctggaggccagcctggaaggtgactcggggaggctgctgtggaaggataactgggtgagcttcatggacaccatgctgcagatgtccatcctgggctcggccaagcacggcctgtacctgcccacccgtgtcaccgccatccacatcgaccctgccacccacaggcagaagctgtacacactgcaggacaaggcccaagtggctgacgtggtggtgagcaggtggctgagggtcacagtggccggaggcgtccacatctccgggctccacactgagtcggccccgcggcggcagcaggagcagcaggtgcccatcctggagaagttttgcttcacttcccacacggaggaggggtgcctgtctgagcgcgctgccctgcaggaggagctgcaactgtgcaaggggctggtgcaggcactgcagaccaaggtgacccagcaggggctgaagatggtggtgcccggactggatggggcccagatcccccgggacccctcacagcaggaactgccccggctgttgtcggctgcctgcaggcttcagctcaacgggaacctgcagctggagctggcgcaggtgctggcccaggagaggcccaagctgccagaggaccctctgctcagcggcctcctggactccccggcactcaaggcctgcctggacactgccgtggagaacatgcccagcctgaagatgaaggtggtggaggtgctggccggccacggtcacctgtattcccgcatcccaggcctgctcagcccccatcccctgctgcagctgagctacacggccaccgaccgccacccccaggccctggaggctgcccaggccgagctgcagcagcacgacgttgcccagggccagtgggatcccgcagaccctgcccccagcgccctgggcagcgccgacctcctggtgtgcaactgtgctgtggctgccctcggggacccggcctcagctctcagcaacatggtggctgccctgagagaagggggctttctgctcctgcacacactgctccgggggcacccctcgggacatgtggccttcctcacctccactgagccgcagtatggccagggcatcctgagccaggacgcgtgggagagcctcttctccagggtgtccgtgcgcctggtgggcctgaagaagtccttctacggctccacgctcttcctgtgccgccggcccaccccgcaggacagccccatcttcctgccggtggacgataccagcttccgctgggtggagtctctgaagggcatcctggctgacgaagactcttcccggcctgtgtggctgaaggccatcaactgtgccacctcgggcgtggtgggcttggtgaactgtctccgccgagagcccggcggaacgctccggtgtgtgctgctctccaacctcagcagcacctcccacgtcccggaggtggacccgggctccgcagaactgcagaaggtgttgcagggagacctggtgatgaacgtctaccgcgacggggcctggggggctttccgccacttcctgctggaggaggacaagcctgaggagccgacggcacatgcctttgtgagcaccctcacccggggggacctgtcctccatccgctgggtctgctcctcgctgcgccatgcccagcccacctgccctggcgcccagctctgcacggtctactacgcctccctcaacttccgcgacatcatgctggccactggcaagctgtcccctgatgccatcccagggaagtggacctcccaggacagcctgctaggtatggagttctcgggccgagacgccagcggcaagcgtgtgatgggactggtgcctgccaagggcctggccacctctgtcctgctgtcaccggacttcctctgggatgtgccttccaactggacgctggaggaggcggcctcggtgcctgtcgtctacagcacggcctactacgcgctggtggtgcgtgggcgggtgcgccccggggagacgctgctcatccactcgggctcgggcggcgtgggccaggccgccatcgccatcgccctcagtctgggctgccgcgtcttcaccaccgtggggtcggctgagaagcgggcgtacctccaggccaggttcccccagctcgacagcaccagcttcgccaactcccgggacacatccttcgagcagcatgtgctgtggcacacgggcgggaagggcgttgacctggtcttgaactccttggcggaagagaagctgcaggccagcgtgaggtgcttggctacgcacggtcgcttcctggaaattggcaaattcgacctttctcagaaccacccgctcggcatggctatcttcctgaagaacgtgacattccacggggtcctactggatgcgttcttcaacgagagcagtgctgactggcgggaggtgtgggcgcttgtgcaggccggcatccgggatggggtggtacggcccctcaagtgcacggtgttccatggggcccaggtggaggacgccttccgctacatggcccaagggaagcacattggcaaagtcgtcgtgcaggtgcttgcggaggagccggaggcagtgctgaagggggccaaacccaagctgatgtcggccatctccaagaccttctgcccggcccacaagagctacatcatcgctggtggtctgggtggcttcggcctggagttggcgcagtggctgatacagcgtggggtgcagaagctcgtgttgacttctcgctccgggatccggacaggctaccaggccaagcaggtccgccggtggagggcccagggcgtacaggtgcaggtgtccaccagcaacatcagctcactggagggggcccggggcctcattgccgaggcggcgcagcttgggcccgtgggcggcgtcttcaacctggccgtggtcttgagagatggcttgctggagaaccagaccccagagttcttccaggacgtctgcaagcccaagtacagcggcaccctgaacctggacagggtgacccgagaggcgtgccctgagctggactactttgtggtcttctcctctgtgagctgcgggcgtggcaatgcgggacagagcaactacggctttgccaattccgccatggagcgtatctgtgagaaacgccggcacgaaggcctcccaggcctggccgtgcagtggggcgccatcggcgacgtgggcattttggtggagacgatgagcaccaacgacacgatcgtcagtggcacgctgccccaggccatggcgtcctgcctggaggtgctggacctcttcctgaaccagccccacatggtcctgagcagctttgtgctggctgagaaggctgcggcctatagggacagggacagccagcgggacctggtggaggccgtggcacacatcctgggcatccgcgacttggctgctgtcaacctggacagctcactggcggacctgggcctggactcgctcatgagcgtggaggtgcgccagacgctggagcgtgagctcaacctggtgctgtccgtgcgcgaggtgcggcaactcacgctccggaaactgcaggagctgtcctcaaaggcggatgaggccagcgagctggcatgccccacgcccaaggaggatggtctggcccagcagcagactcagctgaacctgcgctccctgctggtgaacccggagggccccaccctgatgcggctcaactccgtgcagagctcggagcggcccctgttcctggtgcacccaatcgagggctccaccaccgtgttccacagcctggcctcccggctcagcatccccacctatggcctgcagtgcacccgagctgcgccccttgacagcatccacagcctggctgcctactacatcgactgcatcaggcaggtgcagcccgagggcccctaccgcgtggccggctactcctacggggcctgcgtggcctttgaaatgtgctcccagctgcaggcccagcagagcccagcccccacccacaacagcctcttcctgttcgacggctcgcccacctacgtactggcctacacccagagctaccgggcaaagctgaccccaggctgtgaggctgaggctgagacggaggccatatgcttcttcgtgcagcagttcacggacatggagcacaacagggtgctggaggcgctgctgccgctgaagggcctagaggagcgtgtggcagccgccgtggacctgatcatcaagagccaccagggcctggaccgccaggagctgagctttgcggcccggtccttctactacaagctcggtgccgctgagcagtacacacccaaggccaagtaccatggcaacgtgatgctactgcgcgccaagacgggtggcgcctacggcgaggacctgggcgcggattacaacctctcccaggtatgcgacgggaaagtatccgtccacgtcatcgagggtgaccaccgcacgctgctggagggcagcggcctggagtccatcatcagcatcatccacagctccctggctgagccacgcgtgagcgtgcgggagggctaggcccgtgcccccgcctgccaccggaggtcactccaccatccccaccccaccccaccccacccccgccatgcaacgggattgaagggtcctgccggtgggaccctgtccggcccagtgccactgccccccgaggctgctagatgtaggtgttaggcatgtcccacccacccgccgcctcccacggcacctcggggacaccagagctgccgacttggagactcctggtctgtgaagagccggtggtgcccgttcccgcaggaactgggctgggcctcgtgcgcccgtggggtctgcgcttggtctttctgtgcttggatttgcatatttattgcattgctggtagagacccccaggcctgtccaccctgccaagactcctcaggcagcgtgtgggtcccgcactctgcccccatttccccgatgtcccctgcgggcgcgggcagccacccaagcctgctggctgcggccccctctcggccaggcattggctcagccngctgagtggggggtcgtgggccagtccccgaggagctgggcccctgcacaggcacacagggcccggccacacccagcggccccccgcacagccacccgtggggtgctgcccttatcgcccggcgccgggcaccaactccatgtttggtgtttgtctgtgtttgtttttcaagaaatgattcaaattgctgcttggattttgaaatttactgtaactgtcagtgtacacgtctggaccccgtttcatttttacaccaatttggtaaaaatgctgctctcagcctcccacaattaaaccgcatgtgatctcccc

SEQ ID NO: 3 - protein, 508 aa, Homo sapiens
IVSTQEKELVQPFSSLFPKVEYIARAGAWAMFLDRPQQWLQLVLLPPALFIPSTENEEQRLASARAVPRNVQPYVVYEEVTNVWINVHDIFYPFPQSEGEDELCFLRANECKTGFCHLYKVTAVLKSQGYDWSEPFSPGEGEQSLTNAIWVNEETKLVYFQGTKDTPLEHHLYVVSYEAAGEIVRLTTPGFSHSCSMSQNFDMFVSHYSSVSTPPCVHVYKLSGPDDDPLHKQPRFWASMMEAAKIFHFHTRSDVRLYGMIYKPHALQPGKKHPTVLFVYGGPQVQLVNNSFKGIKYLRLNTLASLGYAVVVIDGRGSCQRGLRFEGALKNQMGQVEIEDQVEGLQFVAEKYGFIDLSRVAIHGWSYGGFLSLMGLIHKPQVFKVAIAGAPVTVWMAYDTGYTERYMDVPENNQHGYEAGSVALHVEKLPNEPNRLLILHGFLDENVHFFHTNFLVSQLIRAGKPYQLQVALPPVSPQIYPNERHSIRCPESGEHYEVTLLHFLQEYL

SEQ ID NO: 4 - protein, 432 aa, Homo sapiens
MHSEQEGQHVQRPCGGKEFGLFEELSEGSFGWVTGIRRMRFKGLAGVDSSLEVVSLLPPRSFSLNSEGAERMATTGTPTADRGDAAATDDPAARFQVQKHSWDGLRSIIHGSRKYSGLIVNKAPHDFQFVQKTDESGPHSHRLYYLGMPYGSRENSLLYSEIPKKVRKEALLLLSWKQMLDHFQATPHHGVYSREEELLRERKRLGVFGITSYDFHSESGLFLFQASNSLFHCRDGGKNGFMVSPGPGCVSPMKPLEIKTQCSGPRMDPKICPADPAFFSFINNSDLWVANIETGEERRLTFCHQGLSNVLDDPKSAGVATFVIQEEFDRFTGYWWCPTASWEEGLKTLRILYEEVDESEVEVIHVPSPALEERKTDSYRYPRTGSKNPKIALKLAEFQTDSQGKIVSTQEKELVQPFSSLFPKVEYIARAG

SEQ ID NO: 5 - protein, 732 aa, Homo sapiens
MERQVLLSEPEEAAALYRGLSRQPALSAACLGPEVTTQYGGQYRTVHTEWTQRDLERMENIRFCRQYLVFHDGDSVVFAGPAGNSVETRGELLSRESPSGSMKAVLRKAGGTGPGEEKQFLEVWEKNRKLKSFNLSVLEKHGPVYEDDCFGCLSWSHSETHLLYVAERKRPKAESFFQTKALDVSASDDEIARLKKPDQPIKGDQFVFYEDWGENMVSKSIPVLCVLDVESGNISVLEGVPENVSPGQAFWAPGDAGVVFVGWWHEPFRLGIRFCTNRRSALYYVDLIGGKCELLSDDSLAVSSPRLSPDQCRIVYLQYPSLIPHHQCSQLCLYDWYTKVTSVVVDVVPRQLGENFSGIYCSLLPLGCWSADSQRVVFDSAQRSRQDLFAVDTQVGTVTSLTAGGSGGSWKLLTIDQDLMVAQFSTPSLPPTLKVGFLPSAGKEQSVLWVSLEEAEPIPDIHWGIRVLQPPPEQENVQYAGLDFEAILLQPGSPPDKTQVPMVVMPHGGPHSSFVTAWMLFPAMLCKMGFAVLLVNYRGSTGFGQDSILSLPGNVGHQDVKDVQFAVEQVLQEEHFDASHVALMGGSHGGFISCHLIGQYPETYRACVARNPVINIASMLGSTDIPDWCVVEAGFPFSSDCLPDLSVWAEMLDKSPIRYIPQVKTPLLLMLGQEDRRVPFKQGMEYYRALKTRNVPVRLLLYPKSTHALSEVEVESDSFMNAVLWLRTHLGS

SEQ ID NO: 6 - DNA, 2374 bp, Homo sapiens
aggcggcagagaggagactatggaacgtcaggtgctgctgagcgagcccgaggaggcggcggctctgtatcggggccttagccgccagcccgcgctgagcgccgcctgcctgggcccggaggtcaccacacagtacggcggccagtaccggacggtgcacactgagtggacccagagggacctggaacgcatggagaacattcgattctgccgccaatacctggtgttccatgacggggactcagtggtgtttgccggacctgcaggcaacagtgtggagacccggggggaactgctgagcagagagtctccttcaggcagcatgaaagctgtgctgcgcaaggctggaggcacgggccctggggaagagaagcagttcctggaggtctgggagaagaaccggaagctcaagagtttcaacctatcagtgctggagaaacatgggcctgtttatgaggatgactgttttggctgcctgtcctggtcgcactcggagacacacttgttgtatgtggcagagaggaagcgccccaaggccgagtccttctttcagaccaaagccttggacgtcagtgccagcgatgatgagatagccaggctgaagaagccagaccaacccatcaagggggatcagtttgtgttttacgaagactggggagaaaacatggtttccaaaagcatccctgtgctctgcgtgctggatgtcgagagtggcaacatctctgtgcttgagggggtccctgagaatgtgtcccctggacaggcattttgggcccctggagatgctggtgtggtgtttgtgggctggtggcatgagcccttccggttgggcatccgcttttgcaccaatcgcaggtcagccctgtattatgtggacctcatcggggggaagtgtgagctcctctcggatgactccctggctgtctcttctccccggctgagcccagaccaatgtcgcattgtctacctgcagtacccatctctgatcccccatcaccaatgcagccagctgtgcctgtatgactggtataccaaggttacctcagtggtggtagatgttgtgcctcggcagctgggagagaacttctctgggatctactgcagccttctgcctttgggatgctggtcagctgacagccagagagtggtctttgactcggctcagcgcagccggcaggacctgtttgctgtggacacccaagtgggcactgtgacctccctcacagctggagggtcaggtgggagctggaagttgctcacaattgaccaggacctcatggtggcacagttttccacacccagcctacctccaaccctgaaagttgggttcctgccttctgcagggaaggagcagtcagtgttgtgggtgtccctggaggaggccgagcccattcccgacatccactggggcatccgggtgctacagccacccccagagcaagagaatgtgcagtatgctggccttgactttgaagcaatcctgctgcagcctggcagccctccagataagacccaagtgcccatggtggtcatgccccacggggggccccattcatcctttgtcactgcctggatgctgttcccagccatgctttgcaagatgggctttgcggtactactagtgaactatcgtggctccacgggctttggccaggacagcatcctctccctcccaggcaatgtgggccaccaggatgtgaaggatgtccagtttgcagtggaacaggtgctccaggaggaacactttgatgcaagccatgtggcccttatgggtggttcccatggtggcttcatttcctgccacttgattggtcagtacccagagacctacagggcctgcgtggcccggaaccccgtgatcaacatcgcctccatgttgggctccactgacatccctgactggtgcgtggtggaggctggctttcctttcagcagtgactgcctgccagacctcagcgtgtgggctgagatgctggacaaatcgcccatcagatacatccctcaggtgaagacaccactgttactgatgttgggccaggaggaccggcgtgtgcccttcaagcagggcatggagtattaccgtgccctcaagacccggaatgtgcctgttcggctcctgctctatcccaaaagcacccacgcattatcagaggtggaggtggagtcagacagcttcatgaatgctgtgctctggctacgcacacacttgggcagctgaagccctgccattctgcatgagctgatcagcctgtgccacacttcgctcttgaggagctcaacggtctggcagggcagcaggaggctttctgggctctggactccacggatgcgtgggcagaggaatgtgggctatgtagtcataataaattaggac

SEQ ID NO: 7 - protein, 710 aa, Homo sapiens
MLSFQYPDVYRDETAVQDYHGHKICDPYAWLEDPDSEQTKAFVEAQNKITVPFLEQCPIRGLYKERMTELYDYPKYSCHFKKGKRYFYFYNTGLQNQRVLYVQDSLEGEARVFLDPNILSDDGTVALRGYAFSEDGEYFAYGLSASGSDWVTIKFMKVDGAKELPDVLERVKFSCMAWTHDGKGMFYNSYPQQDGKSDGTETSTNLHQKLYYHVLGTDQSEDILCAEFPDEPKWMGGAELSDDGRYVLLSIREGCDPVNRLWYCDLQQESSGIAGILKWVKLIDNFEGEYDYVTNEGTVFTFKTNRQSPNYRVINIDFWDPEESKWKVLVPEHEKDVLEWIACVRSNFLVLCYLHDVKNILQLHDLTTGALLKTFPLDVGSIVGYSGQKKDTEIFYQFTSFLSPGIIYHCDLTKEELEPRVFREVTVKGIDASDYQTVQIFYPSKDGTKIPMFIVHKKGIKLDGSHPAFLYGYGGFNISITPNYSVSRLIFVRHMGGILAVANIRGGGEYGETWHKGGILANKQNCFDDFQCAAEYLIKEGYTSPKRLTINGGSNGGLLVAACANQRPDLFGCVIAQVGVMDMLKFHKYTIGHAWTTDYGCSDSKQHFEWLVKYSPLHNVKLPEADDIQYPSMLLLTADHDDRVVPLHSLKFIATLQYIVGRSRKQSNPLLIHVDTKAGHGAGKPTAKVIEEVSDMFAFIARCLNVDWIP

SEQ ID NO: 8 - DNA, 2562 bp, Homo sapiens
atgctgtccttccagtaccccgacgtgtaccgcgacgagaccgccgtacaggattatcatggtcataaaatttgtgacccttacgcctggcttgaagaccccgacagtgaacagactaaggcctttgtggaggcccagaataagattactgtgccatttcttgagcagtgtcccatcagaggtttatacaaagagagaatgactgaactatatgattatcccaagtatagttgccacttcaagaaaggaaaacggtatttttatttttacaatacaggtttgcagaaccagcgagtattatatgtacaggattccttagagggggaggccagagtgttcctggaccccaacatactgtctgacgatggcacagtggcactccgaggttatgcgttcagcgaagatggtgaatattttgcctatggtctgagtgccagtggctcagactgggtgacaatcaagttcatgaaagttgatggtgccaaagagcttccagatgtgcttgaaagagtcaagttcagctgtatggcctggacccatgatgggaagggaatgttctacaactcataccctcaacaggatggaaaaagtgatggcacagagacatctaccaatctccaccaaaagctctactaccatgtcttgggaaccgatcagtcagaagatattttgtgtgctgagtttcctgatgaacctaaatggatgggtggagctgagttatctgatgatggccgctatgtcttgttatcaataagggaaggatgtgatccagtaaaccgactctggtactgtgacctacagcaggaatccagtggcatcgcgggaatcctgaagtgggtaaaactgattgacaactttgaaggggaatatgactacgtgaccaatgaggggacggtgttcacattcaagacgaatcgccagtctcccaactatcgcgtgatcaacattgacttctgggatcctgaagagtctaagtggaaagtacttgttcctgagcatgagaaagatgtcttagaatggatagcttgtgtcaggtccaacttcttggtcttatgctacctccatgacgtcaagaacattctgcagctccatgacctgactactggtgctctccttaagaccttcccgctcgatgtcggcagcattgtagggtacagcggtcagaagaaggacactgaaatcttctatcagtttacttcctttttatctccaggtatcatttatcactgtgatcttaccaaagaggagctggagccaagagttttccgagaggtgaccgtaaaaggaattgatgcttctgattaccagacagtccagattttctaccctagcaaggatggtacgaagattccaatgttcattgtgcataaaaaaggcataaaattggatggctctcatccagctttcttatatggctatggcggcttcaacatatccatcacacccaactacagtgtttccaggcttatttttgtgagacacatgggtggtatcctggcagtggccaacatcagaggaggtggcgaatatggagagacgtggcataaaggtggtatcttggccaacaaacaaaactgctttgatgactttcagtgtgctgctgagtatctgatcaaggaaggttacacatctcccaagaggctgactattaatggaggttcaaatggaggcctcttagtggctgcttgtgcaaatcagagacctgacctctttggttgtgttattgcccaagttggagtaatggacatgctgaagtttcataaatataccatcggccatgcttggaccactgattatgggtgctcggacagcaaacaacactttgaatggcttgtcaaatactctccattgcataatgtgaagttaccagaagcagatgacatccagtacccgtccatgctgctcctcactgctgaccatgatgaccgcgtggtcccgcttcactccctgaagttcattgccacccttcagtacatcgtgggccgcagcaggaagcaaagcaaccccctgcttatccacgtggacaccaaggcgggccacggggcggggaagcccacagccaaagtgatagaggaagtctcagacatgtttgcgttcatcgcgcggtgcctgaatgtcgactggattccataaacagttttcgtgcttcctcctgacagcgacagaaaacctcaagggctttcccacgttgacaccaagaaaccactgggcataatgcttccccacgggaacattattcctgcactcacaggctacagttgaacagaactgccgtgggaattttatcttttttaggcttctcctttttagcaaggccttggtgtttctttttccaccctgtctaggcacatgtggttttttggtgttttttttaagggcatgttgggataaatagctaaatggcaacaaacacattgtgaatattagattgctgaattaaggatcatagtcgggcatacttatttatatccataacctctatatctttaaataaatgtgagaactgttctcatggagaagacttctttgcaacaataataaatgttatttaagaatgaaaaa

SEQ ID NO: 9 - protein, 421 aa, Homo sapiens
MAATLILEPAGRCCWDEPVRIAVRGLAPEQPVTLRASLRDEKGALFQAHARYRADTLGELDLERAPALGGSFAGLEPMGLLWALEPEKPLVRLVKRDVRTPLAVELEVLDGHDPDPGRLLCQTRHERYFLPPGVRREPVRVGRVRGTLFLPPEPGPFPGIVDMFGTGGGLLEYRASLLAGKGFAVMALAYYNYEDLPKTMETLHLEYFEEAMNYLLSHPEVKGPGVGLLGISKGGELCLSMASFLKGITAAVVINGSVANVGGTLRYKGETLPPVGVNRNRIKVTKDGYADIVDVLNSPLEGPDQKSFIPVERAESTFLFLVGQDDHNWKSEFYANEACKRLQAHGRRKPQIICYPETGHYIEPPYFPLCRASLHALVGSPIIWGGEPRAHAMAQVDAWKQLQTFFHKHLGGREGTIPSKV

SEQ ID NO: 10 - DNA, 1568 bp, Homo sapiens
caggtctgaattcaaaatggcctcatctcctgctgtccttcgagcgtcccggctgtaccaatggagcctgaagagttcggcgcagttcctggggtctccacagctgaggcaggttggtcagatcattagggttcctgctcggatggcggcgacgctgatcctggagcctgcgggccgctgctgctgggacgaaccggtgcgaatcgccgtgcgcggcctagccccggagcagccggtcacgctgcgcgcgtccctgcgcgacgagaagggcgcgcttttccaggcccacgcgcgctaccgcgccgacactcttggcgagctggacctggagcgcgcgcccgcgctgggcggcagcttcgcggggcttgagcccatggggctgctctgggccttggagcccgagaaacctttggtgcggctggtgaagcgcgacgtgcgaacgcccttggccgtggagctggaggtgctggatggccacgaccccgaccccgggcggctgctgtgccagacgcggcacgagcgctacttcctcccgcccggggtgcggcgcgagccggtgcgcgtgggccgggtgcgaggcacgctcttcctgccgccagaacctgggccctttcctgggattgtggacatgttcggaactggaggtggcctgctggagtatcgggctagtctgctggctgggaagggttttgctgtgatggctctggcttattataactatgaagacctccccaagaccatggagacgctccatctggagtactttgaagaagccatgaactacttgctcagtcatcccgaggtaaaaggtccaggagttgggctgcttggaatttccaaagggggtgagctctgcctttccatggcctctttcctgaagggcatcacggctgctgtcgtcatcaacggctctgtggccaatgttgggggaaccttacgctacaagggcgagaccctgccccctgtgggcgtcaacagaaatcgcatcaaggtgaccaaagatggctatgcagacattgtggatgtcctgaacagccctttggaaggacctgaccagaagagcttcattcctgtggaaagggcagagagcaccttcctgttcctggtaggtcaggatgaccacaactggaagagtgagttctatgctaatgaggcctgtaaacgcttgcaggcccatgggaggagaaagccccagatcatctgttacccagagacagggcactatattgagcctccttacttccccctgtgtcgggcttccctgcatgccttggtgggcagtcctattatctggggaggggagcccagggctcatgccatggctcaggtggatgcttggaaacaactccagactttcttccacaaacacttgggtggccgcgaggggacaatcccatcaaaagtgtaaattttatttgatcatgtggcctctctgttgctaatctctcctggaaacatctgccacatttagtgtgtgtatgtgtattcattcttttgtttttaataactaaagttttttcccctcattattaaaatgaatttaccagtaaaaaaaaaaaaaaaaaaaa

SEQ ID NO: 11 - protein, 430 aa, Homo sapiens
GGLRVVSPFPLCQPAGEPSRGKMRSSCVLLTALVALATYYVYIPLPGSVSDPWKLMLLDATFRGAQQVSNLIHYLGLSHHLLALNFIIVSFGKKSAWSSAQVKVTDTDFDGVEVRVFEGPPKPEEPLKRSVVYIHGGGWALASAKIRYYDELCTAMAEELNAVIVSIEYRLVPKVYFPEQIHDVVRATKYFLKPEVLQKYMVDPGRICISGDSAGGNLAAALGQQFTQDASLKNKLKLQALIYPVLQALDFNTPSYQQNVNTPILPRYVMVKYWVDYFKGNYDFVQAMIVNNHTSLDVEEAAAVRARLNWTSLLPASFTKNYKPVVQTTGNARIVQELPQLLDARSAPLIADQAVLQLLPKTYILTCEHDVLRDDGIMYAKRLESAGVEVTLDHFEDGFHGCMIFTSWPTNFSVGIRTRNSYIKWLDQNL

SEQ ID NO: 12 - DNA, 4116 bp, Homo sapiens
cggaggtctccgggtggtatcgccctttcctctttgccagcccgctggcgagccgagccggggcaagatgaggtcgtcctgtgtcctgctcaccgccctggtggcgctggccacctattacgtctacatcccgctgcctggctccgtgtccgacccctggaagctgatgctgctggacgccactttccggggtgcacagcaagtgagtaacctgatccactacctgggactgagccatcacctgctggcactgaattttatcattgtttcttttggcaaaaaaagcgcgtggtcttctgcccaagtgaaggtgaccgacacagactttgatggtgtggaagtcagagtgtttgaaggccctccgaagcccgaagagccactgaaacgcagcgtcgtttatatccacggaggaggctgggccttggcaagtgcaaaaatcaggtattatgatgagctgtgtacagcaatggctgaggaattgaatgctgtcattgtttccattgaatacaggctagttccaaaggtttattttcctgagcaaattcatgatgttgtacgggccacaaagtatttcctgaagccagaagtcttacagaagtatatggttgatccaggcagaatttgcatttctggtgacagtgctggtggaaatctggctgctgcccttggacaacagtttactcaagatgccagcctaaaaaataagctcaaactacaagctttaatttatccagttcttcaagctttagattttaacacaccatcttatcagcaaaatgtgaacaccccaatcctgccccgctatgtcatggtgaagtattgggtggactacttcaaaggcaactatgactttgtgcaggcaatgatcgttaacaatcacacttcacttgatgtggaagaggctgctgctgtcagggcccgtctaaactggacatccctcttgcctgcatccttcacaaagaactacaagcctgttgtacagaccacaggcaatgccaggattgtccaggagcttcctcagttgctggatgcccgctccgccccactcattgcagaccaggcagtgctgcagctcctcccaaagacctacattctgacgtgtgagcatgatgtcctcagagacgatggcatcatgtatgccaagcgtttggagagtgccggtgtggaggtgaccctggatcactttgaggatggctttcacggatgtatgattttcactagctggcccaccaacttctcagtgggaatccggactaggaatagttacatcaagtggctagatcaaaacctgtaaaggagcaaaacttccagaagcctcgagcccctcttgacctcctacacctgctttggaaagacatgcactttttagttgactaattcttcctcccattcccctctacttgcgagttatggaatttctattccataactgaagtctttatgataacctaatttttaaaaatgaatttgactaacttaagtgcaaaacatgtaaatttggttcccagagtgggccaatctctctgttcttgttatcttagccaactatactcatacctacagctacagaaagcaggactaggaactggaaataactttgggtcctgccttcattaggacgttctttttagaagcagttcttccagctctggatcatagagtgacctttaataagttaaaaaaacgaggactccttaattctgctagagttaaccttgagttcagagcagtattaaatgcgtgcactttcaggtcagtactggggaccaagtaccctctggtcttttgtgaatggatggttttgtttcctatgggaattttggcaaaggttttctggaaagaacaagtttctcaaaggactttcttcctctagaatgttcattttatgagatcgctatctgtaagtccagttggattacaggaatacttgaaagttactttctaccactattagaaaatatgaagtcgcatgcactggatatctatatatcattaggtttttgttgtgtttttggttatgctgtcccccttctccttggggagatatttgggagcaaacttatttagatttagagtaaacttttcattatagagcaagtaaaaacagacaaatgaaacaacctagtgtttcacataaaaatacttctgacataaagtaccaagagcagtgtgaatatacttggcatagtcaaaaaagaaaatacatttaatattagttaaaaattgttaaaaatacctttagaaggtctagtctattattgaaaactcaattttttcacttatatggctttaaaatggagctattttgctacaatataatgtattgtttatttttttaagttatttaatgttaatatacatagctagacttaaggtttttcagaaagatgtccataataaatattaaaaacaatggtatttttaaaaaaactgccttagggttttaaaaccttccctacagttataaccacgtgtaattttgtggaaatgatataacagctattaatactactataacataggcataaatattttcgtgtttatatgcatatacaagttaaaataattagaaactatgactgcgcctagtaaagtcatctaggtttatagttcagtagcttaggcaaggcacacactgctcatctccgctttttagggtcagaggaacacaagctcatgttctgagtgaagggcgtacactggcacctggtgttgcctagatcccccatctcctccttccagccaggtctggaagtttcaacagcccaagcttaacttcatgtaaagtcttcactgccagtgggaacatctttgacacaacaagacactccaattgtgatttgagttgaggatctctgcctgccttcctgccgtccttccttcttccccgatccatgctacttttaggggctgcggagagcagcagcagagctgagtaatgatacagggcaccacggagagaaagtagaaccatttcactcctgggaagatggggtatttcccacttccagcaacgaaataacaaatgaaaagttgcatacttattgatgtattgtatgagccagtagcattttatgtacaaaacagaagtcaatgcaacagtatgtatgtgtgcctgtgtgtgtataaaaataaccattgaagctaacttgctaatgtacttaggcaagccacttcccatctctgggcctcgtctttcctccctctaaaatcaaagagctgaattatgtgatccttgaggtctcttccacttataataccaactgtcttgtcagactggcaaattatattggcctctccttatgtggtggtttttttggtaggtcatagttccttatacacggacacctgcatcatcgaaggtctttttttcctaaaaaaaaaaaatgggattttagttcttattctgtgataactatcctcctcatataatactattctttttgacaccatttgaaggaaccaatatttggaccttattttgaggttgtctgtctcgaagaaaaagaaaataaaatgtataggcagggttccttcaattggcattttccccagaattgtgagccaaagcctatagtaattgcagacagcaaatgattccggatctctaaaaggctctctcagatgaaaagggagtaaaggaaaaaagagggaggtcaaccactgtttctgataatgtacttgagtttcattgttcttttagtttgtattcttataaaaaatgtttacactctgcagattgattttttttttttagtactgtggctttcttttcctattttatgaaaaaaatgataatctttttgtaaaattgtctgtgaaatataaacattaatatataaagaaaaaccttgaagtgctgtatagtgaagtataaattaatgttttattgatttgtgaagaatttaagactattatataattatcttggtggatctattttatgcatgaccttttaacctttgactttgcttatttcccactacgaaggggaaggtagattttatgaatgattttaatagcaaatatattttataaagtgaaaatccagtgtggaggtagcaaagcatctatctattctgaatcatgtttggaaataaaattgctccatctggg

SEQ ID NO: 13 - protein, 132 aa, Homo sapiens
MRAINIADELPRSRARKLADEQLSSVIQDMAVRQHLLTNLVEVDGRFVWRVNLDALTQHLDKILAFPQRQESYLGPTLFLLGGNSQFVHPSHHPEIMRLFPRAQMQTVPNAGHWIHADRPQDFIAAIRGFLV

SEQ ID NO: 14 - DNA, 1588 bp, Homo sapiens
agcttgcaagcatgctccgctggacccgagcctggaggctcccgcgtgagggactcggcccccacggccctagcttcgcgagggtgcctgtcgcacccagcagcagcagcggcggccgagggggcgccgagccgaggccgcttccgctttcctacaggcttctggacggggaggcagccctcccggccgtcgtctttttgcacgggctcttcggcagcaaaactaacttcaactccatcgccaagatcttggcccagcagacaggccgtgctgacggtggatgctcgtaaccacggtgacagcccccacagcccagacatgagctacgagatcatgagccaggacctgcaggaccttctgccccagctgggcctggtgccctgcgtcgtcgttggccacagcatgggaggaaagacagccatgctgctggcactacagagggtgagccgcccatgtctggggcctcctcccattcagtatataccctgagggccctgcaggcaacctgggactcacatgatcgttggatgaccaagttcaggctccaggagccatgcctgagactccctatgtctgcctaagactggtcccagttcggttctctcccacagccagagctggtggaacgtctcattgctgtagatatcagcccagtggaaagcacaggtgtctcccactttgcaacctatgtggcagccatgagggccatcaacatcgcagatgagctgccccgctcccgtgcccgaaaactggcggatgaacagctcagttctgtcatccaggacatggccgtgcggcagcacctgctcactaacctggtagaggtagacgggcgcttcgtgtggagggtgaacttggatgccctgacccagcacctagacaagatcttggctttcccacagaggcaggagtcctacctcgggccaacactctttctccttggtggaaactcccagttcgtgcatcccagccaccaccctgagattatgcggctcttccctcgggcccagatgcagacggtgccgaacgctggccactggatccacgctgaccgcccacaggacttcatagctgccatccgaggcttcctggtctaagagttgctggcaagaagatggccgggcgtggtggctcatgcctgtaattccagcactttgggaggctaaggcgggaggatgacttgaggccaggagttggagaccagcctggccaacatggtgaaaccctgtctctactaaaaatacaaaaattagcctggcgtggtggtgcacacctgtaatcccagctactctggaggctgaggcaggagaatcacttgaaccctggaggcagaggttgcaatgagccgagatcacaccactacactccagcctaggcaacagagcaagactctgtctcaaaaaaaacaaaacaaaaaggaggcacaaaaccccaggcttcaagtctctgcagcctgctccacatttgggcacagaaggactcagacaggcactgtgtgggcacgaggttttacaggggtggtcagacctcaggctttaatgaataaagacactactccccaaaaaaaaaaaaaaaaaaaaaaaaaaaa

SEQ ID NO: 15 - protein, 306 aa, Homo sapiens
MAVARLAAVAAWVPCRSWGWAAVPFGPHRGLSVLLARIPQRAPRWLPACRQKTSLSFLNRPDLPNLAYKKLKGKSPGIIFIPGYLSYMNGTKALAIEEFCKSLGHACIRFDYSGVGSSDGNSEESTLGKWRKDVLSIIDDLADGPQILVGSSLGGWLMLHAAIARPEKVVALIGVATAADTLVTKFNQLPVELKKEVEMKGVWSMPSKYSEEGVYNVQYSFIKEAEHHCLLHSPIPVNCPIRLLHGMKDDIVPWHTSMQVADRVLSTDVDVILRKHSDHRMREKADIQLLVYTIDDLIDKLSTIVN

SEQ ID NO: 16 - DNA, 2568 bp, Homo sapiens
aactacgaagatggcggttgcgcgcttggcagctgtggcggcctgggtaccttgtcggagctggggctgggcagccgtccccttcggtccccaccgtggcctcagcgtgctgcttgcacggatacctcagcgggcgccacggtggctcccagcttgtagacaaaagacgtcactctcattccttaatcgaccagaccttccaaacctggcttataagaagctaaaaggcaaaagtccaggaattatcttcatccctggctatctttcttatatgaatggtacaaaagcgttggcgattgaggagttttgcaaatctctaggtcacgcctgcataaggtttgattactcaggagttggaagttcagatggtaactcagaggaaagcacactggggaaatggagaaaagatgttctttctataattgatgacttagctgatgggccacagattcttgttggatctagcctcggagggtggcttatgcttcatgctgcaattgcacgaccagagaaggtcgtggctcttattggtgtagctacagctgcagataccttagtgacaaagtttaatcagcttcctgttgagctaaaaaaggaagtagagatgaaaggtgtgtggagcatgccatcaaaatactctgaagaaggagtttataacgttcagtacagtttcattaaagaagctgaacatcactgcttgttacatagcccaattcctgtgaactgccccataagattgctccatggcatgaaggatgacattgtaccttggcatacatcaatgcaggttgccgatcgagtactcagcacagatgtggatgtcatcctccgaaaacacagtgatcaccgaatgagggaaaaagcagacattcaacttcttgtttacactattgatgacttaattgataagctctcaactatagttaactagtatcacatgtttagttggtatgtaaactaatgtatccagaagattggaagagggataagaaatgaaagatcctgatactttaggcttttccctttcctctattttgtaaatataagatgagtattatttaatgatgtatttgcataagtaatgcaaattgtgaagaaggaccagctgctgtttagaaaattttctccttccttctgtccttgattttttttcattaaagtatttcctttttttaattcaagaaaagtttacctttcttatgcttatgttagctatgccagctcttaattgcatccttttctaattaggattattaataaagcgtgaatattttgttttttattatagacagaaatttgtaacattacttctgatttgaaaatgcaattcacaaaatatagggaaatttttattgaataaatttgaaatgatggagaaatttcagaagcataataaagttcacaataaggataatactttatataatgtataaagtatatataatataatatatatgttatataaactgcacattatattcaaacttaaaattgagctttttttttaaaggcccaaaattgtacagtgatacaaggagctatttctaaaatttggcttatgtataatatatttaaatggggaatttcatctaaagcaatgatgtagtatttttaatattctgattggtaaaattaaagaggaaattaatctttatatattatttcttgcagaaacattcattattttattaatattgccctaagtacaactaggcaagtgattgccacctaaatcagaagacgttctaaagtcagtaagaaagtgtgaaatgctagtataaaggttattttttttctttcctaaataactaaagtgaggtgtagattgagccttgatattatttagttaatgttttttattaattaattttggctggactttatttagcttgattaggttattatctgtcaaaccttttaagttgacaacatgactcatatatatacatgtgtataagatgagcatgtgtcaaagacttattcgactcattaatgaggaaaccagcagatagtaaacctggttcaaagtacaattcaagaaactgagtatttatgggcattgaagaaaaaatgttgagataaaattgctgtgcagaaaaaagtgttaatgaagccgacctgactacttaaccttagagacctgctttacaaggttggcccttgattggcatctgggaacttggagttcagggggcttccaccattcccagaactgatcaaagtagcttactatatctaaactgtaaaacaatatagtttctcctgaacacctgctttccttctgggagtctggaattttggtatgtgccaggcagagactacctttgtgaccagctcccagtaaaaaccccaggcactcagtctctaacaagcttttctggttgacagtgtttcacaagtgctgttacaactggttgctgggagaattaagctcatcctctgtgattccactggcggaggattcttggaagcttgcacttagtttcccctgacttcaccccatgtgtcttttttcctttgctgattttgttttgtatcctttcactgtaataaatcatggccgtgagc

SEQ ID NO: 17 - protein, 230 aa, Homo sapiens
  MISC_FEATURE (35)..(72): Xaa is any Amino Acid
MCGNNMSTPLPAIVPAARKATAAVIFLHGLGDTGXXXXXXXXXXXXXXXXXXXXXXXXXXXXXXXXXXXXXXFDIIGLSPDSQEDESGIKQAAENXXXXXXXXXXXXXXXXXXXXXXXXXGGALSLYTALTTQQKLAGVTALSCWLPLRASFPQGPIGGANRDISILQCHGDCDPLVPLMFGSLTVEKLKTLVNPANVTFKTYEGMMHSSCQQEMMDVKQFIDKLLPPID

SEQ ID NO: 18 - protein, 166 aa, Homo sapiens
  MISC_FEATURE (3)..(8): Xaa is any Amino Acid
MNXXXXXXFDIIGLSPDSQEDESGIKQAAENXXXXXXXXXXXXXXXXXXXXXXXXXGGALSLYTALTTQQKLAGVTALSCWLPLRASFPQGPIGGANRDISILQCHGDCDPLVPLMFGSLTVEKLKTLVNPANVTFKTYEGMMHSSCQQEMMDVKQFIDKLLPPID

SEQ ID NO: 19 - DNA, 2417 bp, Homo sapiens
  misc_feature (1)..(2417): n is any nucleotide
cttccttccgcttgcgctgtgagctgaggcggtgtatgtgcggcaataacatgtcaaccccgctgcccgccatcgtgcccgccgcccggaaggccaccgctgcggtgattttcctgcatggattgggagatactgggcacggatgggcagaagcctttgcaggtatcagaagttcacatatcaaatatatctgcccgcatgcgcctgttaggcctgttnancattaaatatgaacnnnnnnnnnnnnnnnnngtttgatattattgggctttcaccagattcacaggaggatgaatctgggattaaacaggcagcagaaaatannnnnnnnnnnnnnnnnnnnnnnnnnnnnnnnnnnnnnnnnnnnnnnnnnnnnnnnnnnnnnnnnnnnnnnnnnggaggagctttatctttatatactgcccttaccacacagcagaaactggcaggtgtcactgcactcagttgctggcttccacttcgggcttcctttccacagggtcctatcggtggtgctaatagagatatttctattctccagtgccacggggattgtgaccctttggttcccctgatgtttggttctcttacggtggaaaaactaaaaacattggtgaatccagccaatgtgacctttaaaacctatgaaggtatgatgcacagttcgtgtcaacaggaaatgatggatgtcaagcaattcattgataaactcctacctccaattgattgacgtcactaagaggccttgtgtagaagtacaccagcatcattgtagtagagtgtaaaccttttcccatgcccagtcttcaaatttctaatgttttgcagtgttaaaatgttttgcaaatacatgccaataacacagatcaaataatatctcctcatgagaaatttatgatcttttaagtttctatacatgtattcttataagacgacccaggatctactatattagaatagatgaagcaggtagcttcttttttctcaaatgtaattcagcaaaataatacagtactgccaccagattttttattacatcatttgaaaattagcagtatgcttaatgaaaatttgttcaggtataaatgagcagttaagatataaacaatttatgcatgctgtgacttagtctatggatttattccaaaattgcttagtcaccatgcagtgtctgtatttttatatatgtgttcatatatacataatgattataatacataataagaatgaggtggtattacattattcctaataatagggataatgctgtttattgtcaagaaaaagtaaaatcgttctcttcaattaatggcccttttattttgggaccaggcttttattttccctgatattatttctatttaatactcttttctctcaagaaaaaaaaaaaagtttgttttttctttattgtccttcatagcaggccaagtattgcctctctgcaatagacagctactgtcaatacatgctgtaatttgacattctgggtcacagatataaggtatttaaaatctatttatgctttatagagaaaccagacattaaaacttcatgcactacttatttcgaattactgtaccttatccaaatttacacctagctattaggatcttcaacccaggtaacaggaataattctgtggtttcatttttctgtaaacaactgaaagaataattagatcatattctagtatgttctgaaatatctttaagactgatcttaaaaactaacttctaagatgatttcatcttctcatagtatagagtttactttgtacacgtttgaaaccaactactgtagaagatgaggaatctattgtaattttttgctttattttcatctgccagtggacttatttgaaattttcactttagtcaaattattttttgtattagtttttgatgcagacataaaaatagcaatcattttaaattgtcaaaatttccagattactggtaaaaattatttgaaaacaaacttatgggtaataaaggctagtcagaaccctataccataaagtgtagttaccatacagattaatatgtagcaaaaatgtatgcttgatatttctcaactgtgttaatttttctgctgtattccagctgaccaaaacaatattaagaatgcatctttataaatgggtgctaattgataatggaaataatttagtaatggactatacaggatgttaataatgaagccatatgtttatgtctggatttaaaaattttaaacaatcatttactatgtcatttttctttaccttgaagaacataaactgttatttcacttctacaaatcagcaagatattatttatggcaagaaatattccattgaaatattgtgctgtaacatgggaaagtgtaaatgtttttcatggtttctatcaatgtgaaataaaatttaattctga